SEQUENCE LISTING (2 sequences)

SEQ ID NO: 1 - DNA, 1514 bp, Homo sapiens
agactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcagcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttc

SEQ ID NO: 2 - DNA, 1492 bp, Mus musculus
acacactctgccttgctcacagaggaggggctgcagccctggccctcatcagaacaatgacactcaggctgctgttcttggctctcaacttcttctcagttcaagtaacagaaaacaagattttggtaaagcagtcgcccctgcttgtggtagatagcaacgaggtcagcctcagctgcaggtattcctacaaccttctcgcaaaggaattccgggcatccctgtacaagggcgtgaacagcgacgtggaagtctgtgtcgggaatgggaattttacctatcagccccagtttcgctcgaatgccgagttcaactgcgacggggatttcgacaacgaaacagtgacgttccgtctctggaatctgcacgtcaatcacacagatatttacttctgcaaaattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaagtgactaccatgaacatgactccccggaggcctgggctcactcgaaagccttaccagccctacgcccctgccagagactttgcagcgtaccgcccctgacagggacccctatccagaagcccgccggctggtacccgtctacctgctcatcatcactgctctggataggaaaggacagcctcatcttcagccggccactttggacctctactgggccaccaatgccaactattttagagtgtctagatctaacatcatgatcatcttgagactctggaatgaatgacagaagcttctatggcaggataaagtctgtgtggcttgacccaaactcaagcttaatacatttattgacttgattggggaagttagagtagagcaatcaaaaagatcattcattcagccttgggaagtcaatttgcaggctcctggatgagccctgccccgttttcacttgccagcacatttcagtcatgtggtgtgatagccaaagatgttttggacagagaagaaaggatagaaaaaccttctctttggctaagttggtgtttggggtggggataggttagagtatagtacttaactatttgaaaaataatgaaaacacttttttcactcatgaaatgagccacttagctcctaaatagtgttttcctgttagtttagaaagttgtggacatatttttttaatgatttctgaccatttttaatcacattgactcatggaatggcctcaaagcaccccccagtgcttctttcctcattcccggtcatgggaactcagtattattaatagtcacaacatgatttcagaactagatagccctcccacaccaagaagaatgtgagaggaagtaaggtcactttatgtaaaaacg

(19) United States
(12) Patent Application Publication
(10) Pub. No.: US 2002/0006403 A1
(43) Pub. Date: Jan. 17, 2002

(54) CD28-specific antibody compositions for use in methods of immunosuppression

(76) Inventors: Xue-Zhong Yu, Seattle, WA (US); Claudio Anasetti, Mercer Is., WA (US)

Correspondence Address: Steven L. Highlander, Fulbright & Jaworski L.L.P., Suite 2400, 600 Congress Avenue, Austin, TX, 78701, US

(21) Appl. No.: 09/738,546

(22) Filed: Dec. 14, 2000

Related U.S. Application Data
(60) Provisional application No. 60/170,857, filed on Dec. 14, 1999.

(51) Int. Cl. A61K 39/395; A61K 38/13; A61K 31/675; A61K 31/573

(52) U.S. Cl. 424/142.1; 514/9; 514/291; 514/49; 514/105; 514/171; 514/250

(57) ABSTRACT

The present invention provides methods for suppressing, reducing or even reversing an immune response. More particularly it concerns anti-CD28 monoclonal antibody compositions and methods for preventing graft-versus-host disease (GVHD), transplant tissue rejection, and treating autoimmune diseases and the like. In particular embodiments, a method of inhibiting an immune response comprises administering an effective amount of a purified anti-CD28 antibody preparation to a subject, wherein the preparation modulates the CD28 receptor thereby inhibiting an immune response.

### RELATED APPLICATION

This application claims priority from U.S. Provisional Patent Application No. 60/170,857 filed Dec. 14, 1999.

### BACKGROUND OF THE INVENTION

1. Field of the Invention

The present invention relates generally to the field of immunology. More particularly, it concerns anti-CD28 monoclonal antibody compositions and methods of preventing graft-versus-host disease. In other embodiments, compositions and methods of the present invention are contemplated for use in marrow and solid organ transplantation and in the treatment of autoimmune diseases.

2. Description of Related Art

While stimulation of the immune systems prevents and controls infection, it can have an adverse physiological effect, as is the case with autoimmune diseases, with rejection of cells and tissues during adoptive immunotherapy and transplants, and with invasions by pathogens. Thus, inhibition of this stimulation can have beneficial therapeutic results.

Cell-mediated immunity occurs when sensitized T cells directly damage cells or release lymphokines that augment the inflammatory reaction. The B cell production of antibodies that bind ‘self’ antigens are referred to as autoantibodies (i.e., self antibodies). An association of an autoantibody with its antigen in intercellular fluid causes cell lysis and autoantibody-induced release of inflammatory mediators. This interaction results in release of inflammatory mediators, induction of the complement pathway, or activation of cytotoxic cells, which can trigger cell lysis. Another mechanism, immune complex disease, involves a reaction between circulating autoantibodies and antigens on the cell surface. This complex becomes deposited in tissues such as the joints, blood vessels, and glomeruli, causing complement to be fixed and subsequent inflammation and tissue damage.

For example, graft-versus-host disease (GVHD) results from donor T cell activation in response to alloantigens expressed by the host. In GVHD, the grafted immune system attacks the host cells. GVHD becomes particularly significant in bone marrow transplantation (BMT), which is frequently used for the treatment of a variety of bone marrow-related disorders and in cancer therapy to replace bone marrow cells lost to chemotherapy and radiation treatment. In severe cases of GVHD, a patient's compromised immune system gives rise to many complications including those in the liver, causing jaundice, in the skin, causing rash, and in the gastrointestinal tract, including diarrhea, anorexia, nausea and vomiting, malabsorption, abdominal pain, ileus, and ascites formation.

The primary determinant of T cell activation is the interaction of T cell receptors (TCR) with antigenic peptides presented by major histocompatibility complex (MHC) molecules on antigen-presenting cells (APC). The costimulatory molecules B7-1 and B7-2 expressed on APC, collectively referred to herein as B7, regulate T cell activation by delivering activation signals through CD28 (Hara et al., 1985; Shahinian et al., 1993; Lenschow et at., 1996) and inhibitory signals through cytotoxic T lymphocyte-associated antigen 4 (CTLA4) (Walunas et al., 1994; Krummel and Allison, 1995; Tivol et al., 1995; Waterhouse et al., 1995; Thompson and Allison, 1997). The importance of the B7:CD28/CTLA4 pathways have been highlighted by studies showing that B7 blockade can suppress GVHD and autoimmunity (Wallace et al., 1996; Blazar et al., 1994; Blazar et al., 1995; Blazar et al., 1996; Miller et al., 1995). Blockade of CTLA4 alone, however, can exacerbate autoimmune disease and enhance anti-tumor immunity (Perrin et al., 1996; Leach et al., 1996).

GVHD remains a major complication of human allogeneic hematopoietic cell transplantation causing high morbidity and mortality (Armitage, 1994; Hansen et al., 1997; Hansen et al., 1998). An effective treatment for the prevention of cell and tissue rejection following transplantation, including GVHD, has not been identified (Saigo and Ryo, 1999). Thus, the ability to prevent GVHD disease, cell and tissue rejection during adoptive immunotherapy and transplantation, as well as the treatment of autoimmune diseases, are highly desirable.

### SUMMARY OF THE INVENTION

The present invention addresses the need for preventing graft-versus-host disease (GVHD), transplant tissue rejection and treating autoimmune diseases. The invention provides in particular embodiments, methods for suppressing, reducing or even reversing an immune response.

In one embodiment, a method of inhibiting an immune response comprises administering to a subject an effective amount of a purified anti-CD28 antibody preparation, wherein the preparation modulates the CD28 receptor thereby inhibiting the immune response. In particular embodiments, inhibiting an immune response with an anti-CD28 antibody preparation is achieved by reversing T cell activation or blocking T cell activation.

In certain embodiments, an anti-CD28 antibody preparation is polyclonal. In other embodiments, the anti-CD28 antibody preparation is monoclonal, wherein the antibody is monovalent or bivalent. In particular embodiments, the antibody is human. In yet other embodiments, the anti-CD28 antibody preparation is chimeric, wherein the chimeric antibody is humanized. In preferred embodiments, the humanized antibody comprises mammalian variable chain regions and human constant chain regions, wherein the mammalian variable chain regions are selected from the group consisting of mouse, rat, hamster, monkey, goat and human.

In certain embodiments, inhibiting an immune response in a subject by administering an effective amount of a purified anti-CD28 antibody preparation, the subject is susceptible to graft-versus-host disease, marrow transplant rejection, organ transplant rejection or tissue transplant rejection. In particular embodiments, a subject may have graft-versus-host disease. In other embodiments, a subject has an autoimmune disease, wherein the autoimmune disease is psoriasis, diabetes mellitus, multiple sclerosis, rheumatoid arthritis, systemic sclerosis, systemic lupus erythematosus, dermatomyositis, polymyositis, Sjogren syndrome, polyarteritis nodosa or vasculitis.

In another embodiment of the invention, administering an anti-CD28 antibody preparation is by injection. In one embodiment, the injection is performed local or regional to the site of immune response. In certain embodiments, the injection site is further defined as thymus, spleen, lymph nodes, bone marrow, tonsils, adenoids or blood stream. In further embodiments, the injection is parenteral, intravenous, intramuscular, subcutaneous, intradermal or intraperitoneal, most preferably intraperitoneal or intravenous. In yet other embodiments, administering an anti-CD28 antibody preparation comprises multiple injections, wherein injections are performed at the same time at different locations or at different times. In embodiments where administering an anti-CD28 antibody preparation is by intraperitoneal or intravenous injection, the injection may be via continuous infusion.

In other embodiments, administering an anti-CD28 antibody preparation by injection further comprises an immunosuppressive agent, wherein the immunosuppressive agent is selected from the group consisting of azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody.

In another embodiment of the present invention, a method of inhibiting an immune response in a subject is provided, comprising the steps of obtaining lymphocyte cells from the subject, contacting the lymphocyte cells with an anti-CD28 antibody preparation and administering the contacted cells to the subject, wherein the preparation reverses T cell activation thereby inhibiting the immune response.

In particular embodiments, the antibody preparation is polyclonal. In other embodiments, the antibody preparation is monoclonal. In certain embodiments, the antibody preparation is monovalent or bivalent. In one embodiment, the antibody is human. In another embodiment, the antibody is chimeric, wherein the chimeric antibody is humanized. In preferred embodiments, the humanized antibody comprises mammalian variable chain regions and human constant chain regions, wherein the mammalian variable chain regions are selected from the group consisting of mouse, rat, hamster, monkey, goat and human.

In particular embodiments of the invention, wherein inhibiting an immune response in a subject comprises obtaining lymphocyte cells from the subject, contacting the lymphocyte cells with an anti-CD28 antibody preparation and administering the contacted cells to the subject, the subject treated is susceptible to graft-versus-host disease, marrow transplant rejection, organ transplant rejection or tissue transplant rejection. In a preferred embodiment, the subject has graft-versus-host disease.

In certain embodiments, inhibiting an immune response in a subject comprises obtaining lymphocyte cells from the subject, contacting the lymphocyte cells with an anti-CD28 antibody preparation and administering the contacted cells to the subject, administering the cells is by injection. In one embodiment, the lymphocyte cells are obtained from thymus, spleen, lymph nodes, bone marrow, tonsils, adenoids or blood stream. In other embodiments, the injection further comprises an immunosuppressive agent, wherein the immunosuppressive agent is selected from the group consisting of azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody.

In yet other embodiments, the subject has psoriasis, diabetes mellitus, multiple sclerosis, rheumatoid arthritis, systemic sclerosis, systemic lupus erythematosus, dermatomyositis, polymyositis, Sjogren syndrome, polyarteritis nodosa or vasculitis.

A further embodiment of the invention involves a method of inhibiting a CD28 mediated response by binding CD28 with a ligand. In the context of the invention, a ligand is broadly defined as a molecule that binds to another molecule. In vivo, the ligand for CD28 is normally either B7-1 or B7-2. When either of these molecules bind the CD28 receptor, a signal is delivered to the T-cell. Nevertheless, the inventors specifically envision that other ligands exist that may bind CD28 without initiating signal transduction. These molecules, for example an antibody, would function to prevent the CD28/B7 interaction and thus prevent full T-cell activation.

A further embodiment of the invention involves a method of inhibiting a CD28 mediated response by blocking CD28 signal transduction. Signal transduction could be blocked, for example by introducing a ligand that binds PI 3-kinase.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B. CTLA4 protects mice from GVHD. (B6×bm12)F1 mice were irradiated (700 cGy) and transplanted with purified CD4⁺ cells from CD28^{+/+} (FIG. 1A) or CD28^{-/-} B6 (FIG. 1B) donors. Irradiated (B6×bm12)F1 mice were injected with PBS alone as no transplant control. Each Ab was injected at 100 μg/mouse every other day for a total of 8 doses. Data are shown from one study for FIG. 1A, and two replicate studies for FIG. 1B.

FIG. 2A and FIG. 2B. Anti-CD28 mAb is more effective in preventing lethal GVHD than CTLA4-Ig or anti-CD28-Fab. (B6×bm12)F1 (FIG. 2A) or (B6×bm1)F1 (FIG. 2B) mice were irradiated (700 cGy) and transplanted with purified CD4⁺ (FIG. 2A) or CD8⁺ (FIG. 2B) cells from B6 mice, respectively. A group of irradiated F1 mice were injected with PBS alone as no transplant controls. Recipients were treated with the antibodies (Abs) indicated from day 0 to day 14, except for anti-CD28 Fab from day 0 to day 16. Data were pooled from four replicate studies for FIG. 2A, and three replicate studies for FIG. 2B.

FIG. 3. Anti-CD28 mAb inhibits donor CD4 T cell expansion and modulates CD28. (B6.Ly55.1×bm12)F1 recipients were transplanted with B6.Ly5.2 CD4⁺ T cells and treated with anti-CD28 mAb, CTLA4-Ig or control Abs. On day 4, recipient splenocytes were stained for expression of Ly5. 1, CD4 and CD28. Top panels show the percentage and absolute number of CD4⁺/Ly5.1⁻ donor T cells, and bottom panels show CD28 expression on gated donor T cells.

FIG. 4. Anti-CD28 mAb inhibits donor T cell expansion, but does not affect on expression of CD25 and CTLA4. CB6F1 mice were transplanted with CD8⁺ cells from CD28^{+/+} or CD28^{-/-} 2C donors and treated with anti-CD28 mAb or control hamster IgG. On day 4, recipient splenocytes were tested for the expression of 1B2 (the clonotypic 2C TCR-specific mAb), CD8, CD25 and CTLA4. Data represent one of three similar studies.

FIG. 5. Anti-CD28 mAb selectively inhibits expansion of 2C T cells and destruction of host B cells in CB6F1 recipients. CB6F1 (L^{d+}) or dm2B6F1 (L^{d-}) mice were transplanted with 2C CD8⁺ cells and treated with control Ab or anti-CD28 mAb. On day 14, splenocytes form each recipient were stained for 1B2, CD8 and B220, and analyzed by 3-color flow cytometry. The values shown are absolute number of each population per spleen. The results present average 1±SD from 2-3 mice per each group.

### DESCRIPTION OF ILLUSTRATIVE EMBODIMENTS

The present invention addresses the need for methods and compositions for preventing graft-versus-host disease (GVHD), transplant tissue rejection and the like and treating autoimmune diseases and the like. The costimulatory molecules B7-1 and B7-2 regulate T cell activation by delivering activation signals through CD28 and inhibitory signals through cytotoxic T lymphocyte-associated antigen 4 (CTLA4). GVHD is caused by activated donor T cells. The inventors have previously demonstrated that CD28-deficient donor T cells induced less-severe GVHD than wild-type donor T cells. In addition, CTLA4-signals attenuate the severity of GVHD, independent of CD28.

The present invention demonstrates that targeting the CD28 receptor with a specific monoclonal antibody (mAb) modulates the receptor in vivo, inhibits donor T cell expansion, and prevents GVHD. The present invention also demonstrates that anti-CD28 mAb directed modulation of the CD28 receptor is more immunosuppressive than methods that block the CTLA4 function or methods that block both CTLA4 and CD28 function. In particular embodiments, the present invention provides anti-CD28 mAb compositions and methods for inhibiting T cell activation. Contemplated in the present invention is a T cell costimulation model in which CD28 signals amplify GVHD, while CTLA4 signals inhibit GVHD. Therefore, selective targeting of CD28 with anti-CD28 mAb is a useful therapeutic strategy for inducing immunological tolerance, rather than blocking the ligands for both CD28 and CTLA4.

A. Treatment Uses of Anti-CD28 Antibodies

Monoclonal antibodies immunoreactive with a CD28 receptor will be useful in preventing various immune related disorders. For example, prevention of transplant rejection, GVHD, and treating autoimmune diseases and the like are contemplated using anti-CD28 mAbs of the present invention. Polyclonal anti-CD28 antibody preparations also are contemplated for use in treating immune related disorders. The following are representative of some of the immune related complications and diseases that may potentially be treated via the present invention.

1. Transplantation Rejections and Graft-Versus-Host Disease

Transplant rejections occur as a consequence of an immune response against the transplanted organ, tissue, or cells. Antigens on the surface of the transplanted material act to signal that it is foreign, and a response ensues. Conversely, GVHD occurs when the graft mounts an immune response against the host, which can happen following a bone marrow transplant or blood transfusions. Lymphocytes in the donor marrow participate in the destruction of host cells through the actions of T-lymphocytes which serve as helper cells in anti-host cell lysis and B-lymphocytes which produce anti-host antibodies. It occurs in approximately 100% of patients receiving an allogeneic transplant depending on the degree of histocompatibility between donor and recipient.

Because immunostimulation occurs in both transplant rejections and GVHD, the anti-CD28 antibody compositions and methods of the present invention can be used as treatments to inhibit an immune response, and thus alleviate or eliminate their destructive outcomes. “Inhibiting an immune response” in the present invention includes, but is not limited to, an ability to suppress, reduce, or reverse, even slightly an immune response.

2. Autoimmune Diseases and Phenomena

There are numerous conditions that qualify as an autoimmune disease. They occur either when the immune system malfunctions and the lymphocytes become sensitized against self tissue cells or when self tissue cells exhibit non-self characteristics such as expression of different antigens. Some of the most common disorders are listed in Table 1, such as rheumatoid arthritis and lupus erythrematosus.

Other autoimmune diseases include: Alopecia Areata, Acquired Hemophilia, Ankylosing Spondylitis, Antiphospholipid Syndrome, Autoimmune Hepatitis, Autoimmune Hemolytic Anemia, Behcet's Disease, Cardiomyopathy, Celiac Sprue Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Discoid Lupus, Essential Mixed Cryoglobulinemia, Fibromyalgia, Fibromyositis, Guillain-Barre, Idiopathic Pulmonary Fibrosis, Idiopathic Thrombocytopenic Purpura, IgA Nephropathy, Juvenile Arthritis, Lichen Planus, Multiple Sclerosis, Myasthenia Gravis, Polyarteritis Nodosa, Polychondritis, Polyglandular Syndromes, Dermatomyositis, Primary Agammaglobulinemia, Primary Biliary Cirrhosis, Psoriasis, Raynaud's Phenomena, Reiter's Syndrome, Sarcoidosis, Stiff-Man Syndrome, Takayasu Arthritis, Temporal Arteritis/Giant Cell Arteritis, Ulcerative Colitis, Uveitis, Vasculitis, and Vitiligo.

TABLE 1
Selected Autoimmune Diseases and the Targets of Their Antibodies

| | Target of Antibody |
|---|---|
| Systemic (Non-Organ-Specific) | |
| Diseases | |
| Goodpasture's Syndrome | Basement membranes |
| Mixed Connective Tissue Disease | Nuclear ribonucleoproteins |
| Polymyositis | Nuclei, Jo-1, PL-7, histadyl-tRNA |
| | synthetase, threonyl-tRNA |
| | synthetase, PM-1, Mi-2 |
| Rheumatic Fever | Myocardium, heart valves, choroid |
| | plexus |
| Rheumatoid Arthritis | γ-Globulin, Epstein-Barr virus- |
| | related antigens, types II and |
| | III collagen |
| Scleroderma | Nuclei, Scl-70, SS-A (Ro), |
| | SS-B (La), centromere |
| Sjögren's Syndrome | γ-Globulin, SS-A (Ro), SS-B (La) |
| Systemic Lupus Erythematosus | DNA, ribonucleoproteins, histones, |
| | nuclear antigens |
| Wegener's granulomatosis | Neutrophils |
| Organ-Specific Diseases | |
| Addison's Disease | Adrenal cells |
| Allergic Rhinitis, Asthma, and | β₂-adrenergic receptors |
| autoimmune abnormalities | |
| Autoimmune hemolytic anemia | Erythrocytes |
| Acquired Hemophilia | Clotting Factor VIII |
| Bullous Pemphigoid | Basement membrane zone of |
| | skin and mucosa |
| Chronic Active Hepatitis | Nuclei of hepatocytes |
| Crohn's Disease | Lymphocytes, plasma cells, |
| | eosinophils |
| Glomerulonephritis | Glomeruli |
| Graves’ Disease | Thyroid-stimulating hormone |
| | (TSH) receptor |
| Hashimoto's Thyroiditis | Thyroglobulin, TSH receptor |
| Idiopathic Hypoparathyroidism | Parathyroid cells |
| Idiopathic Neutropenia | Neutrophils |
| Idiopathic Thrombocytopenic Purpura | Platelets |
| Insulin-resistant Diabetes with | Insulin receptor |
| acanthosis nigricans | |
| Insulin-resistant Diabetes with ataxia- | Insulin receptor |
| telangiectasia | |
| Juvenile Insulin-dependent Diabetes | Pancreatic islet cells |
| Ménière's Disease | Type II collagen |
| Myasthenia Gravis | Acetylcholine receptors |
| Osteosclerosis | Type II collagen |
| Pemphigus | Intercellular substance of skin and |
| | mucosa |
| Pernicious Anemia | Gastric parietal cells, vitamin B₁₂ |
| | binding site of intrinsic factor |
| Premature Ovarian Failure | Interstitial cells, corpus |
| | luteum cells |
| Primary Biliary Cirrhosis | Mitochondria |
| Spontaneous infertility | Sperm cells |

The present invention is understood to include anti-CD28 compositions that act to inhibit a T cell immune response, and thus serve to prevent or allay the progression of autoimmune diseases and associated phenomena. Furthermore, methods of treating autoimmune diseases using these compositions also are contemplated.

3. Sepsis

Sepsis can be caused by many different infectious agents and microbial organisms that may or may not be involved directly with bloodstream infection. It is a condition characterized by an inflammatory response. The term “sepsis” as used herein broadly refers to conditions known as sepsis, septic shock, systemic inflammatory response syndrome (SIRS), and multiple organ dysfunction syndrome (MODS). Because these conditions are caused by an inflammatory response of the immune system, the compositions and methods of the present invention can be employed as preventative and as therapeutic treatments to inhibit an immune response.

B. Nucleic Acids

The present invention provides anti-CD28 monoclonal antibody (mAb) compositions and methods that inhibit T cell immune responses. In particular embodiments of the present invention, a gene encoding a CD28 polypeptide is used to obtain CD28 polypeptide for use in generating anti-CD28 mAbs. The preparation and purification of CD28 polypeptides (Section C) and anti-CD28 antibody preparations (Section D) using CD28 polypeptides are described below.

Thus, in certain embodiments of the present invention, genes encoding CD28 are provided. It is contemplated in the present invention, that a polynucleotide encoding a CD28 polypeptide is expressed in prokaryotic cells and the CD28 polypeptides purified for use in generating anti-CD28 antibodies. In other embodiments, a polynucleotide encoding a CD28 polypeptide is expressed in eukaryotic cells either in vivo or cell culture.

Genes for the mouse (Gross et al., 1990; genbank acc. M34563), cat (genbank acc. U57754), dog (Pastori et al., 1994; genbank acc. L22178;), sheep (Chaplin et al., 1999; genbank acc. AF092739), chicken (genbank acc. AWO61436), cow (Parsons et al., 1996; genbank acc. X93304), rabbit (Isono and Seto, 1995; genbank acc. D49841;) and rat (Clark and Dalman, 1992; genbank acc. X55288;), CD28 molecules have been identified. The present invention is not limited in scope to these genes, however, as one of ordinary skill in the art could, using these nucleic acids, readily identify related homologs in various other species (e.g., monkey, gibbon, chimp, ape, baboon, pig, sheep, goat, human and other species). The finding of hamster and mouse homologs for this gene makes it likely that other species more closely related to humans will, in fact, have a homolog as well.

In addition, it should be clear that the present invention is not limited to the specific nucleic acids disclosed herein. As discussed below, a “CD28 gene” may contain a variety of different bases and yet still produce a corresponding polypeptides that is functionally indistinguishable, and in some cases structurally, from the human (SEQ ID NO:1) and mouse (SEQ ID NO:2) genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of CD28.

1. Nucleic Acids Encoding CD28

The present invention provides polynucleotides encoding CD28 polypeptides, for use as an antigen to generate anti-CD28 antibodies. In certain instances, it may be desirable to express CD28 polynucleotides encoding a particular antigenic CD28 polypeptide domain or sequence to be used in generating anti-CD28 antibodies. Nucleic acids according to the present invention may encode an entire CD28 gene, a domain of CD28, or any other fragment of the CD28 sequences set forth herein. The nucleic acid may be derived from genomic DNA, i.e., cloned directly from the genome of a particular organism. In preferred embodiments, however, the nucleic acid comprises complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression.

It also is contemplated that a given CD28 from a given species may be represented by natural variants that have slightly different nucleic acid sequences but, nonetheless, encode the same protein (see Table 2 below). In addition, it is contemplated that a given CD28 from a species may be generated using alternate codons that result in a different nucleic acid sequence but encodes the same protein.

As used in this application, the term “a nucleic acid encoding a CD28” refers to a nucleic acid molecule that has been isolated free of total cellular nucleic acid. The term “functionally equivalent codon” is used herein to refer to codons that encode the same amino acid, such as the six codons for arginine or serine (Table 2, below), and also refers to codons that encode biologically equivalent amnino acids, as discussed in the following pages.

TABLE 2

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of a mouse or hamster CD28. Sequences that are essentially the same as those set forth in a mouse or hamster CD28 gene may also be functionally defined as sequences that are capable of hybridizing to a nucleic acid segment containing the complement of a mouse or hamster CD28 polynucleotide under standard conditions.

The DNA segments of the present invention include those encoding biologically functional equivalent CD28 proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

2. Oligonucleotide Probes and Primers

Naturally, the present invention also encompasses DNA segments that are complementary, or essentially complementary to the sequences of a CD28 gene. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of a mouse polynucleotide and a hamster polynucleotide under relatively stringent conditions such as those described herein. Such sequences may encode the entire CD28 protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to CD28 or, more particularly, homologs of CD28 from other species. The existence of a murine homolog strongly suggests that other homologs of the human CD28 will be discovered in species more closely related, and perhaps more remote, than mouse or hamster. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Polypeptides

For the purposes of the present invention a CD28 protein or peptide used as an antigen may be a naturally-occurring CD28 protein that has been extracted using protein extraction techniques well known to those of skill in the art.

In alternative embodiments, the CD28 protein peptide or antigen may be a synthetic peptide. In still other embodiments, the peptide may be a recombinant peptide produced through molecular engineering techniques. The present section describes the methods and compositions involved in producing a composition of CD28 proteins for use as antigens in the present invention.

1. CD28 Polypeptides

CD28 protein encoding genes or their corresponding cDNA can be inserted into an appropriate cloning vehicle for the production of CD28 proteins as antigens for the present invention. In addition, sequence variants of the polypeptide can be prepared. These may, for instance, be minor sequence variants of the polypeptide that arise due to natural variation within the population or they may be homologues found in other species. They also may be sequences that do not occur naturally, but that are sufficiently similar that they function similarly and/or elicit an immune response that cross-reacts with natural forms of the polypeptide. Sequence variants can be prepared by standard methods of site-directed mutagenesis such as those described below in the following section.

Another synthetic or recombinant variation of a CD28-antigen is a polyepitopic moiety comprising repeats of epitopic determinants found naturally on CD28 proteins. Such synthetic polyepitopic proteins can be made up of several homomeric repeats of any one CD28 protein epitope; or can comprise of two or more heteromeric epitopes expressed on one or several CD28 protein epitopes.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide may be identified by an empirical approach in which portions of the gene encoding the polypeptide are expressed in a recombinant host, and the resulting proteins tested for their ability to elicit an immune response. For example, the polymerase chain reaction (PCR) can be used to prepare a range of cDNAs encoding peptides lacking successively longer fragments of the C-terminus of the protein. The immunogenic activity of each of these peptides then identifies those fragments or domains of the polypeptide that are essential for this activity. Further experiments in which only a small number of amino acids are removed or added at each iteration then allows the location of other antigenic determinants of the polypeptide. Thus, the polymerase chain reaction, a technique for amplifying a specific segment of DNA via multiple cycles of denaturation-renaturation, using a thermostable DNA polymerase, deoxyribonucleotides and primer sequences is contemplated in the present invention (Mullis, 1990; Mullis et al., 1992).

Another embodiment for the preparation of the polypeptides according to the invention is the use of peptide mimetics. Mimetics are peptide-containing molecules that mimic elements of protein secondary structure. Because many proteins exert their biological activity via relatively small regions of their folded surfaces, their actions can be reproduced by much smaller designer (mimetic) molecules that retain the bioactive surfaces and have potentially improved pharmacokinetic/dynamic properties (Fairlie et al., 1998).

The underlying rationale behind the use of peptide mimetics is that the peptide backbone of proteins exists chiefly to orient amino acid side chains in such a way as to facilitate molecular interactions, such as those of antibody and antigen. However, unlike proteins, peptides often lack well defined three dimensional structure in aqueous solution and tend to be conformationally mobile. Progress has been made with the use of molecular constraints to stabilize the bioactive conformations. By affixing or incorporating templates that fix secondary and tertiary structures of small peptides, synthetic molecules (protein surface mimetics) can be devised to mimic the localized elements of protein structure that constitute bioactive surfaces. Methods for mimicking individual elements of secondary structure (helices, turns, strands, sheets) and for assembling their combinations into tertiary structures (helix bundles, multiple loops, helix-loop-helix motifs) have been reviewed (Fairlie et al., 1998; Moore, 1994).

Methods for predicting, preparing, modifying, and screening mimetic peptides are described in U.S. Pat. No. 5,933,819 and U.S. Pat. No. 5,869,451 (each specifically incorporated herein by reference). It is contemplated in the present invention, that peptide mimetics will be useful in screening modulators of an immune response. In specific embodiments, a modulator is an inhibitor of an immune response. Peptide mimetics in the context of the present invention could be used to screen for modulators of B7 and CD28.

Modifications and changes may be made in the structure of a gene and still obtain a functional molecule that encodes a protein or polypeptide with desirable characteristics. The following is a discussion based upon changing the amino acids of a protein to create an equivalent, or even an improved, second-generation molecule. The amino acid changes may be achieved by changing the codons of the DNA sequence, according to the following data.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventor that various changes may be made in the DNA sequences of genes without appreciable loss of their biological utility or activity. Table 2 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982).

It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

2. Synthetic Polypeptides

The present invention also describes CD28 proteins and related peptides for use as antigens in various embodiments of the present invention. In certain embodiments, the synthesis of a CD28 domain or peptide fragment is considered. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide of the invention is inserted into an expression vector, transformed or transfected into an appropriate host cell and cultivated under conditions suitable for expression.

3. CD28 Polypeptide Purification

CD28 polypeptides of the present invention are used as antigens for the preparation of anti-CD28 monoclonal antibodies. Thus, certain aspects of the present invention concern the purification, and in particular embodiments, the substantial purification, of the CD28 polypeptide that is described herein above. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater -fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

D. CD28 Antibodies

In another aspect, the present invention provides antibody compositions that are immunoreactive with a CD28 molecule of the present invention, or any portion thereof. The inventors demonstrate, in the present invention, that bivalent monoclonal anti-CD28 antibodies are immunosuppressive, which is contrary to previous anti-CD28 studies. The CD28 molecule is a homodimer, i.e. it is composed by two identical glycosylated polypeptides joined together by disulfide bonds. CD28 binds CD80 or CD86 expressed on a variety of cells that present antigen to T cells. Engagement of CD28 by its natural ligands induces intracellular activation leading to cytokine expression, proliferation, and cytotoxic function (Jung et al., 1987). Similarly, bivalent anti-CD28 antibodies cross-link the CD28 homodimer and induce T cell activation (Tan, 1993). In contrast, monovalent anti-CD28 antibodies cannot cross-link the CD28 homodimer and are unable to induce T cell activation and, furthermore, they are capable to inhibit T cell responses in vitro.

Investigators have not tested the immunosuppressive activity of anti-CD28 antibodies because bivalent antibodies activate rather than block T cell function in vitro, and monovalent antibodies are difficult to make. Against all predictions, the inventors have discovered that the same bivalent anti-CD28 antibody, which activates T cells in vitro, is immunosuppressive in vivo. It was observed that the immunosuppressive activity of the bivalent anti-CD28 antibody is mediated by blocking and internalization of the CD28 molecules. After anti-CD28 binding, most CD28 molecules are transported inside the T cell, so that there are no longer surface CD28 molecules to bind CD80 and CD86 and activate the T cell. Furthermore, the few CD28 molecules that persist on the cell surface are blocked by the antibody and do not function.

An antibody can be a polyclonal or a monoclonal antibody. An antibody may also be monovalent or bivalent. A prototype antibody is an immunoglobulin composed by four polypeptide chains, two heavy and two light chains, held together by disulfide bonds. Each pair of heavy and light chains forms an antigen binding site, also defined as complementarity-determining region (CDR). Therefore, the prototype antibody has two -CDRs, can bind two antigens, and because of this feature is defined bivalent. The prototype antibody can be split by a variety of biological or chemical means. Each half i-of the antibody can only bind one antigen and, therefore, is defined monovalent. In a preferred embodiment, an anti-CD28 antibody is a bivalent monoclonal antibody. Means for preparing and characterizing antibodies are well known in the art (see, e.g., Howell and Lane, 1988).

Peptides corresponding to one or more antigenic determinants of a CD28 polypeptide of the present invention also can be prepared. Such peptides should generally be at least five or six amino acid residues in length, will preferably be about 10, 15, 20, 25 or about 30 amino acid residues in length, and may contain up to about 35-50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

The identification and preparation of epitopes from primary amino acid sequences on the basis of hydrophilicity is taught in U.S. Pat. No. 4,554,101 (Hopp), incorporated herein by reference. Through the methods disclosed in Hopp, one of skill in the art would be able to identify epitopes from within an amino acid sequence such as a CD28 polypeptide sequence.

Numerous scientific publications have also been devoted to the prediction of secondary structure, and to the identification of epitopes, from analyses of amino acid sequences (Chou & Fasman, 1974a; Chou & Fasman, 1974b; Chou & Fasman, 1978a; Chou & Fasman, 1978b; Chou & Fasman, 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PEPPLOT® (Brutlag et al, 1990; Weinberger et al., 1985), and other new programs for protein tertiary structure prediction (Fetrow & Bryant, 1993). Another commercially available software program capable of carrying out such analyses is MACVECTOR (IBI, New Haven, Conn.).

In further embodiments, major antigenic determinants of a CD28 polypeptide may be identified by an empirical approach in which portions of the gene encoding the polypeptide are expressed in a recombinant host, and the resulting proteins tested for their ability to elicit an immune response. For example, PCR can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTS system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR cloning methodology.

The use of such small peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

1. Anti-CD28 Antibody Generation

The present invention provides monoclonal antibody compositions that are immunoreactive with a CD28 molecule. As detailed above, in addition to antibodies generated against a full length CD28 polypeptide, antibodies also may be generated in response to smaller constructs comprising epitopic core regions, including wild-type and mutant epitopes. In other embodiments of the invention, the use of anti-CD28 single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” CD28 antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-CD28 humanized or chimeric antibody can be genetically engineered to comprise a CD28 antigen binding site of a given of molecular weight and biological lifetime, as long as the antibody retains its CD28 antigen binding site.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), chimeras and the like. Methods and techniques of producing the above antibody-based constructs and fragments are well known in the art (U.S. Pat. No. 5,889,157; U.S. Pat. No. 5,821,333; U.S. Pat. No. 5,888,773, each specifically incorporated herein by reference).

U.S. Pat. No. 5,889,157 describes a humanized B3 scFv antibody preparation. The B3 scFv is encoded from a recombinant, fused DNA molecule, that comprises a DNA sequence encoding humanized Fv heavy and light chain regions of a B3 antibody and a DNA sequence that encodes an effector molecule. The effector molecule can be any agent having a particular biological activity which is to be directed to a particular target cell or molecule. Described in U.S. Pat. No. 5,888,773, is the preparation of scFv antibodies produced in eukaryotic cells, wherein the scFv antibodies are secreted from the eukaryotic cells into the cell culture medium and retain their biological activity. It is contemplated that similar methods for preparing multi-functional anti-CD28 fusion proteins, as described above, may be utilized in the present invention.

Means for preparing and characterizing antibodies also are well known in the art (See, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic CD28 polypeptide composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate asuppressor cell activity. Such BRMs include, but are not limited to, Cimetidine (CIM; 1200 mg/d) (SmithKline Beecham, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate mAbs.

For production of rabbit polyclonal antibodies, the animal can be bled through an ear vein or alternatively by cardiac puncture. The removed blood is allowed to coagulate and then centrifuged to separate serum components from whole cells and blood clots.

The serum may be used as is for various applications or else the desired antibody fraction may be purified by well-known methods, such as affinity chromatography using another antibody, a peptide bound to a solid matrix, or by using, e.g., protein A or protein G chromatography. mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified CD28 polypeptide, peptide or domain, be it a wild-type or mutant composition. The immunizing composition is administered in a manner effective to stimulate antibody producing cells.

The methods for generating monoclonal antibodies (mAbs) generally begin along the same lines as those for preparing polyclonal antibodies. Rodents such as mice and rats are preferred animals, however, the use of rabbit, sheep or frog cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8,×63-Ag8.653, NSI/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. HAT medium, a growth medium containing hypoxanthine, aminopterin and thymidine, is well known in the art as a medium for selection of hybrid cells. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmnunoassays, enzyme immunoassays, cytotoxicity assays, plaque assays, dot immunobinding assays, and the like.

The selected hybridomas then would be serially diluted and cloned into individual antibody-producing cell lines, which clones can then be propagated indefinitely to provide mAbs. The cell lines may be exploited for mAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide mAbs in high concentration. Second, the individual cell lines could be cultured in vitro, where the mAbs are naturally secreted into the culture medium from which they can be readily obtained in high concentrations.

mAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in, for example, E. coli.

E. CD28 Ligands

Methods for the construction or eluciation of ligands to block T cell activation by the CD28 complex are well known in the art. U.S. Pat. No. 5,525,503 discloses compositions and methods of blocking T cell signal transduction by introducing into a T cell a peptide comprising a PI 3-kinase-binding-sequence which decreases the association of PI 3-kinase with CD28. This methodology is further applicable to the prevention of the onset of GVHD through the prevention of the delivery of the CD28 signal to the nucleus. In addition to the antibody preparations disclosed herein, it is envisioned that other ligands, both natural and synthetic may be used in the prevention of the CD28/B7 interaction necessary for T-cell activation. Molecules capable of binding to CD28 without inducing signal transduction are known or can be readily derived based upon the knowledge of one of ordinary skill in combination with structural data available regarding the CD28 molecule. (Holdorf, 1999; Linsley, 1995)

F. Therapeutic Formulations and Routes of Anti-CD28 Antibody Administration

The present invention discloses the compositions and methods involving monoclonal antibody preparations reactive with a CD28 polypeptide, that inhibit T cell stimulation of the immune system. Where clinical applications are contemplated, it will be necessary to prepare the compositions of the present invention as pharmaceutical compositions, i.e., in a form appropriate for in vivo applications. Generally, this will entail preparing compositions that are essentially free of pyrogens, as well as other impurities that could be harmful to humans.

One will generally desire to employ appropriate salts and buffers to render compositions stable. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, buffers, dispersion media, coatings, antibacterial and antifungal agents, isotonic and absorption delaying agents and the like. The use of such media and agents for pharmaceutically active substances is well known in the art. Except insofar as any conventional media or agent is incompatible with the anti-CD28 compositions and methods of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target CD28 receptor is available via that route. Administration includes intravenous injection, intradermal injection, peritoneal injection, intraperitoneal injection, subcutaneous injection, oral, nasal, buccal, rectal, vaginal or topical. Such compositions would normally be administered as pharmaceutically acceptable compositions, described above.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Anti-CD28 preparations of the present invention, when injected, may be performed local or regional to the site of immune inhibition. In addition, such injections sites may include the thymus, spleen lymph nodes, bone marrow, tonsils, adenoids and blood stream. Injections of anti-CD28 antibody preparations may be parenteral, intravenous, intramuscular, subcutaneous, intradermal, peritoneal, intraperitoneal, or any other mode of injection suitable to deliver said anti-CD28 antibody preparations. Injection may be performed at the same time at different locations, at different times, via continuous infusion, or in combination with other methods of anti-CD28 delivery (e.g., suppository).

Additional formulations which are contemplated suitable as modes of administration include suppositories and, in some cases, oral or nasal formulations. For suppositories, traditional binders and carriers may include, for example, polyalkalene glycols or triglycerides: such suppositories may be formed from mixtures containing the active ingredient in the range of about 0.5% to about 10%, preferably about 1 to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10 to about 95% of active ingredient, preferably about 25 to about 70%.

One also may use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

G. Combined Therapy with Anti-CD28 and Traditional Treatment

In many therapies, it will be advantageous to provide more than one functional therapeutic. Such “combined” therapies may have particular import in treating aspects of autoimmune diseases/phenomena and tissue/organ rejections. Thus, one aspect of the present invention utilizes at least one CD28 mAb immunoreactive with a CD28 receptor for treatment of immunostimulation, while a second therapy also is provided.

1. Inhibitors of Immunostimulation

The invention involves compositions and methods that effect inhibition of immunostimulation. As used herein, the terms “inhibition of immunostimulation” or “to inhibit immunostimulation” refer to an ability to suppress or reduce, even slightly, an immune response. An immune response can be evidenced by a number of characteristics including, but not limited to, production of lymphokines or cytokines, release of lymphokines or cytokines, proliferation of lymphocytes, activation of lymphocytes, complement fixing, induction of the complement cascade, production of antibodies, release of antibodies, release of inflammatory mediators, and binding of T cells to a T-cell receptor. In particular embodiments, the present invention contemplates the inhibition of an immune response, wherein an anti-CD28 antibody preparation is administered to a subject and modulates the CD28 receptor resulting in the inhibition of an immune response. In specific embodiments, the inhibition of an immune response is by reversing T cell activation or by blocking T cell activation via the modulation of the CD28 receptor.

The following are immunosuppressive agents contemplated for use in combination with anti-CD28 antibody preparations of the present invention. Azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody. This list is not comprehensive however, and any other immunosuppressive agents that have a combined immune inhibitory effect with anti-CD28 antibody preparations of the present invention are considered.

The immunosuppressive agents may precede or follow an anti-CD28 antibody preparation by intervals ranging from minutes to weeks. In embodiments where the immunosuppressive agents and anti-CD28 antibody preparation are applied separately to the cell, one would generally ensure that a significant period of time did not expire between the time of each delivery, such that the agent and antibody would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the immunosuppressive agent is “A” and the anti-CD28 antibody preparation is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Agents or factors suitable for use in a combined therapy are any chemical compound or treatment method with immunosuppressive activity; therefore, the term “immunosuppressive agent” that is used throughout this application refers to an agent with immunosuppressive activity. Immunosuppressive agents such as azathioprine and cyclosporin are employed in transplant procedures to treat and prevent rejections. Compounds or methods used to treat GVHD include corticosteroids such as prednisone, antithymocyte globulins, cyclosporine A, cyclophosphamide, and methotrexate. Thalidomide is occasionally employed in combination with one of the previously mentioned corticosteroids to treat GVHD, and it is contemplated that thalidomide could also be used in combination with the compounds of the present invention. Similarly, patients with autoimmune diseases are administered immunosuppressant medications like corticosteroids, cyclophosphamide, and azathioprine.

In the treatment of sepsis, other agents or compounds that would be useful for use in a combination therapy with the compounds and methods of the claimed invention include antibiotics such as cephalosporin, fluoroquinolones, penicillins, carbapenems, β-lactams-β-lactamase inhibitors, ampicillin, vancomycin, metronidazole, clindamycin, and trovafloxacin, as well as with corticosteroids, vasopressor agents, vasoconstrictors, and beta agonists.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

The inventors propose that local, regional delivery of at least anti-CD28 mAb will be a very efficient method for delivering a therapeutically effective compound to counteract the clinical disease. Similarly, the immunosuppressive agent may be directed to a particular, affected region of the subject's body. Alternatively, systemic delivery of compounds and/or the agents may be appropriate in certain circumstances, for example, where extensive tissue damage has occurred.

H. Genetic Constructs and Their Delivery to Cells

Within certain embodiments, expression vectors can be employed to express various CD28 genes to produce large amounts of the CD28 polypeptide product, which then can be purified and used as an antigen in the present invention, or to vaccinate animals to generate antisera or monoclonal antibodies. In other embodiments, an expression vector can be used to express anti-CD28 antibodies, single chain antibodies, chimeras and the like. This section provides a description of the production of genetic constructs and their delivery into cells for protein expression.

1. Genetic Constructs

Within certain embodiments expression vectors can be employed to express a various genes to produce large amounts of a CD28 polypeptide product, which can then be purified and used as an antigen in the present invention or to vaccinate animals to generate antisera or monoclonal antibodies with which further studies may be conducted. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the CD28 products are also provided, as is an element that links expression of the drug selection markers to expression of the polypeptide.

a. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter refers to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the cell. Thus, where a human cell is used, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Table 3 and Table 4 list several inducible elements/promoters which may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a -gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct. Enhancer/promoter elements contemplated for use with the present invention include but are not limited to Immunoglobulin Heavy Chain, Imunoglobulin Light, Chain T-Cell Receptor, HLA DQ α and DQ β, β-Interferon, Interleukin-2, Interleukin-2 Receptor, MHC Class II 5, MHC Class II HLA-DRα, β-Actin, Muscle Creatine Kinase, Prealbumin (Transthyretin), Elastase I, Metallothionein, Collagenase, Albumin Gene, α-Fetoprotein, τ-Globin, β-Globin, e-fos, c-HA-ras, Insulin, Neural Cell Adhesion Molecule (NCAM), α1-Antitrypsin, H2B (TH2B) Histone, Mouse or Type I Collagen, Glucose-Regulated Proteins (GRP94 and GRP78), Rat Growth Hormone, Human Serum Amyloid A (SAA), Troponin I (TN I), Platelet-Derived Growth Factor, Duchenne Muscular Dystrophy, SV40, Polyoma, Retroviruses, Papilloma Virus, Hepatitis B Virus, Human Immunodeficiency Virus, Cytomegalovirus, Gibbon Ape Leukemia Virus. Inducible promoter elements and their associated inducers are listed in Table 3 and enhancer/promoter elements are listed in Table 4 below.

TABLE 3

| Element | Inducer |
|---|---|
| MT II | Phorbol Ester (TPA), Heavy metals |
| MMTV (mouse mammary tumor | Glucocorticoids |
| virus) | |
| β-Interferon | poly(rI)X, poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV4O Large T Antigen |
| Proliferin | Phorbol Ester-TPA |
| Tumor Necrosis Factor | FMA |
| Thyroid Stimulating Hormone | Thyroid Hormone |
| α Gene | |
| Insulin E Box | Glucose |

TABLE 4
ENHANCER/PROMOTER

Immunoglobulin Heavy Chain
Immunoglobulin Light Chain
T-Cell Receptor
HLA DQ a and DQ b
b-Interferon
Interleukin-2
Interleukin-2 Receptor
MHC Class II 5
MHC Class II HLA-DRa
b-Actin
Muscle Creatine Kinase
Prealbumin (Transthyretin)
Elastase I
Metallothionein
Collagenase
Albumin Gene
a-Fetoprotein
t-Globin
b-Globin
e-fos
c-HA-ras
Insulin
Neural Cell Adhesion Molecule (NCAM)
al-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV4O
Polyoma
Retroviruses
Papilloma Virus
Hepatitis B Virus
Human Immunodeficiency Virus
Cytomegalovirus
Gibbon Ape Leukemia Virus

In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). Adeno-associated viruses are also useful in this context (Ridgeway, 1988; Baichwal and Sugden, 1986; Hermonat and Muzycska, 1984). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

b. Selectable Markers

In certain embodiments of the invention, the cells contain nucleic acid constructs for the production of CD28 antigens, such a cell may be identified by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

c. Multigene constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

2. Delivery of Genetic Constructs

In order to express the proteins from the expression constructs, the CD28 nucleic acids need to be delivered into a cell. There are a number of ways in which nucleic acids may introduced into cells. Several methods, including viral and non-viral transduction methods, are outlined below.

a. Adenovirus

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

Generation and propagation of adenovirus vectors, which are replication deficient, depend on a unique helper cell line, designated 293, which was transformed from human embryonic kidney cells by Ad5 DNA fragments and constituitively expresses El proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra Kb of DNA. Combined with the approximately 5.5 Kb of DNA that is replaceable in the E1 and E3 regions, the maximum capacity of the current adenovirus vector is under 7.5 Kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permnissive for human adenovirus. Such cells include, e.g., Vero cells or other monkey embryonic mesenchymal or epithelial cells. As stated above, the preferred helper cell line is 293.

The nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

The typical adenoviral vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the CD28 gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992).

b. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via asialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

c. Adeno-Associated Virus (AAV)

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, pl9 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

The terminal repeats of the AAV vector can be obtained by restriction endonuclease digestion of AAV or a plasmid such as p201, which contains a modified AAV genome (Samulski et al. 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

d. Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) and herpes viruses may be employed. They offer several attractive features for various mammalian cells (Friedmann, 1989; Ridgeway, 1988; Baichwal and Sugden, 1986; Coupar et al., 1988; Horwich et al., 1990).

In vitro studies of hepatitis B viruses showed the virus retained the ability for helper-dependent packaging and reverse transcription despite the deletion of up to 80% of its genome (Horwich et al., 1990), suggesting that large portions of the genome could be replaced with foreign genetic material. The hepatotropism and persistence (integration) are particularly attractive properties for liver-directed gene transfer. Chang et al., 1991, recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

e. Non-viral vectors

Several non-viral methods for the transfer of expression constructs into cultured mammalian cells are contemplated by the present invention. These include calcium phosphate precipitation (Graham and Van Der Eb, 1973; Chen and Okayama, 1987) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al., (1984) successfully injected polyomavirus DNA in the form of calcium phosphate precipitates into liver and spleen of adult and newborn mice demonstrating active viral replication and acute infection. Benvenisty and Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring naked DNA expression constructs into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a -high voltage discharge to generate an electrical current, which in turn provides the motive force (Yang et al., 1990). The microprojectiles used have consisted of biologically inDcqt substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growvth factor (EGF) has also been used to deliver genes to squamous carcinoma cells (Myers, EPO 0273085).

I. Cell Culture

In order to produce large quantities of an CD28 protein from a cell transfected with an expression construct as described herein above, it may be necessary to grow the cell in culture for a period of time to allow protein production to occur. Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

Animal cells can be propagated in vitro in two modes: as non-anchorage dependent cells growing in suspension throughout the bulk of the culture or as anchorage-dependent cells requiring attachment to a solid substrate for their propagation (i.e. a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent T-cells.

As would be evident to one of ordinary skill in the art, that the CD28 proteins also may be expressed in a variety of organisms including but not limited to Saccharomyces cerevisiae, filamentous fungi, and E. coli. Methods for expressing cloned genes in Saccharomyces cerevisiae are generally known in the art (see, “Gene Expression Technology,” Methods in Enzymology, Vol. 185, Goeddel (ed.), Academic Press, San Diego, CA, 1990 and “Guide to Yeast Genetics and Molecular Biology,” Methods in Enzymology, Guthrie and Fink (eds.), Academic Press, San Diego, Calif., 1991; which are incorporated herein by reference).

Filamentous fungi (e.g., strains of Aspergillus) also may be used to express the proteins of the present invention. Methods for expressing genes and cDNAs in cultured mammalian cells and in E. coli is discussed in detail in Sambrook et al. (Molecular Cloning: A Laboratory Manual, Second Edition, Cold Spring Harbor, N.Y., 1989; which is incorporated herein by reference). As would be evident to one skilled in the art, one could express the protein of the instant invention in other host cells such as avian, insect and plant cells using regulatory sequences, vectors and methods well established in the literature.

Large scale suspension culture of cells in stirred tanks is a common method for production of recombinant proteins. Two suspension culture reactor designs are in wide use—the stirred reactor and the airlift reactor. The stirred design has successfully been used on an 8000 liter capacity for the production of interferon. Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Mice. C57BL/6 (B6), B6.C-H2^{bm12} (bm12), B6.C-H2^{bm1} (bm1), (B6×BALB/c)F1 (CB6F1), BALB/c H2-dm (dm2) and B6.SJL-Ly5^{a} Ptprc^{a} Pep3^{b} (136.Ly5.1) mice were purchased from the Jackson Laboratory (Bar Harbor, Me.). (B6×bm12)F1, (B6×bm1)F1 and (B6×dm2)F1 (dm2B6F1) mice were bred at Fred Hutchinson Cancer Research Center (FHCRC, Seattle, Wash.). Founders for 2C transgenic mice were supplied from Dr. Dennis Y. Loh (Nippon Roche Research Center, Kamakur-shi, Japan). Homozygous B6 CD28^{-/-} mice were obtained from Dr. Craig Thompson (Shahinian et al., 1993). 2C CD28^{-/-} mice were generated by intercrossing 2C to CD28^{-/-}. All the mice used in this report were housed in microisolator cages.

T-cell purification and transplantation. The inventors' transplant protocol for T-cell purification and transplantation has been described in detail (Yu et al., 1998). CD4⁺ or CD8⁺ T cells were purified by positive selection using a magnetic cell separation system (Miltenyi Biotech Inc., Auburn, Calif.). To avoid the possibility of graft rejection, F1 mice were used as recipients in all studies. (136×bm12)F1 or (B6×bm1)F1 mice were exposed to 700 cGy of irradiation (⁶⁰Co source) at 20 cGy per min. CB6F1 or dm2B6F1 mice were irradiated with 750 cGy. One million of purified CD4⁺ or CD8⁺ cells from B6 donor were transplanted into via the tail vein into irradiated (B6×bm12)F1 or (B6×bm1)F1 recipients respectively. In some studies, recipients with the congenic marker Ly5.1 were utilized in order to distinguish donor from host cells. Irradiated CB6F1 or dm2B6F recipients were transplanted with purified CD8⁺ cells from 2C donors. The number of 2C T cells injected ranged from 6 to 15×10⁶ per mouse, and equal cell number was transplanted into each recipient for the same individual study.

Preparation and administration of Abs. Both anti-CD28 (37.51) and anti-CTLA4 (9H10) are hamster IgG and provided by Dr. James Allison (University of California, Berkeley, Calif.). Murine CTLA4-Ig and control L6-Ig were obtained from Dr. Robert Peach (Bristol-Myers Squibb, Princeton, N.J.). Control hamster Ig was purchased from IGN Pharmaceuticals, Inc. (Aurora, Ohio). Anti-CD28 Fab fragments were obtained by papain digestion. All the antibodies (Abs), unless indicated, were injected intraperitoneally at 100 μg/dose every other day for 14 days starting on day 0.

Flow cytometry. To detect donor CD4 or CD8 cells, splenocytes were isolated from the recipients and stained with mabs specific for Ly5.1 (A20-1.7, mouse IgG2a; American Type Culture Collection, Rockville, Md.) and CD4 (GK1.5) or CD8 (53-6.7). For detection of 2C donor cells, mAbs specific for CD8 and 2C TCR (1B2) were used. The 1B2 hybridoma was kindly obtained from Dr. Loh (Sha et al., 1998), and FITC-conjugated 1B2 was prepared in the inventors' laboratory. Other mAbs used in this study included: anti-B220 (RA3-6B2), anti-CD28 (37.51), anti-CD25 (7D4), anti-CTLA4 (9H10), mouse anti-hamster IgG (192.1) and isotype control Abs. All mAbs used for FACS analysis, unless indicated, were obtained from PharMingen (San Diego, Calif.). Flow cytometric analysis was performed on a FACScan using CELLQuest software (Becton Dickinson, San Jose, Calif.).

2C cells engraft in irradiated F1 mice. The inventors have established a model where CD8⁺ 2C cells cause GVHD in sublethally irradiated CB6 recipients. CD8⁺ 2C cells were purified (>95% IB2⁺) by positive selection with MACS, and 10⁵, 10⁶, or 10⁷ cells were transplanted into each irradiated (750 cGy) allogenic CB6 recipient by tail vein injection. CB6 mice, that were not transplanted, were used as irradiation controls. Irradiation was delivered by a ⁶⁰Co source at the rate of 20 cGy per min. Mice were nursed in microisolator cages, and provided low bacterial diet and autoclaved water. Donor CD8⁺/1B2⁺ 2C cells appeared in spleen, lymph nodes and blood of all CB6 recipients transplanted with 10⁷ cells. In contrast, IB2⁺ cells could not be visualized in CB6 mice transplanted with 10⁵ or 10⁶ 2C cells.

2C cells induce GVHD in F1 mice. Further studies investigated whether, in CB6 mice transplanted with 10⁷ 2C cells, engraftment led to disease. All mice lost 6-8% total body weight following irradiation. CB6 recipients took 5 wk to regain the original weight and resume growth while controls began to recover at 2 wk. CB6 recipients appeared much less active in their cages than irradiation CB6 controls or syngenic B6 controls. No mouse died spontaneously through day 120, the latest time point observed so far in this type of study. One mouse from each group was sacrificed on days 35, 42, 49, and 56 after transplantation, and thymus, lymph nodes, spleen, blood and bone marrow were collected. An equal number of axillary, brachial, inguinal, and mesenteric lymph nodes was collected from each mouse at each time point. Bone marrow cells were obtained from one femur and one tibia per mouse.

Double-positive thymocytes are GVHD targets for 2C cells. The total number of thymocytes was decreased by 97-99% in CB6 recipients compared to syngenic recipients or irradiation controls. No CD8⁺/IB2⁺ 2C cells could be detected in the thymus of B6 recipients but they constituted a large proportion of the residual cells in the thrones of CB6 recipients. The CD4 and CD8 double-positive population had disappeared from the thymus of CB6 recipients, while it was the predominant cell population in syngenic recipients and irradiation controls. Thus, not only were 2C cells visualized in the thymus of allogeneic CB6 recipients, but their presence was associated with profound pathology of the thymus that was reduced to a remnant and devoid of immature double positive thymocytes.

Allogenic B and T cells are GVHD targets for 2C cells. B and T cells were markedly depleted in spleen and lymph nodes of CB6 recipients compared to B6 recipients and irradiation controls. Light microscopy of H and E stained tissues showed a profound decrease in the number and size of follicles in the spleen of CB6 mice. The architecture of the lymph nodes was disrupted, and few lymphocytes were left in the midst of fibrous tissue. Marrow cellularity was decreased by 70%, and mature B cells were absent from the marrow of CB6 mice. Thus, transplantation of 2C cells in CB6 recipients but not B6 recipients is associated with profound pathology of all the hematopoietic organs examined. CD8⁺/1B2⁺ 2C cells appeared in the blood of both B6 and CB6 recipients, but not in irradiated CB6 controls. At all time points, host CD8⁺/1B2⁻ T cells and B cells were significantly reduced in CB6 recipients compared to controls. Thus, B and T cells were not only depleted in the lymphoid organs but also in the blood of allogeneic CB6 recipients

Biological factors that affect alloreactivity in vivo. The 2C transplantation model of non lethal GVHD mediated by 2C cells, that the inventors have described here, provides the unique advantage of allowing the inventors to follow the fate of the alloreactive cells after treatment with antimetabolites that could not be done if mice died rapidly. Thymocyte and peripheral T and B cell numbers provide a quantitative measure of the intensity of GVHD. Thus, the inventors have developed the tools needed to evaluate antimetabolites that are expected to induce T cell tolerance by T cell deletion.

### Example 2

### Determining the Role of CTLA4-signals on the Development of GVHD

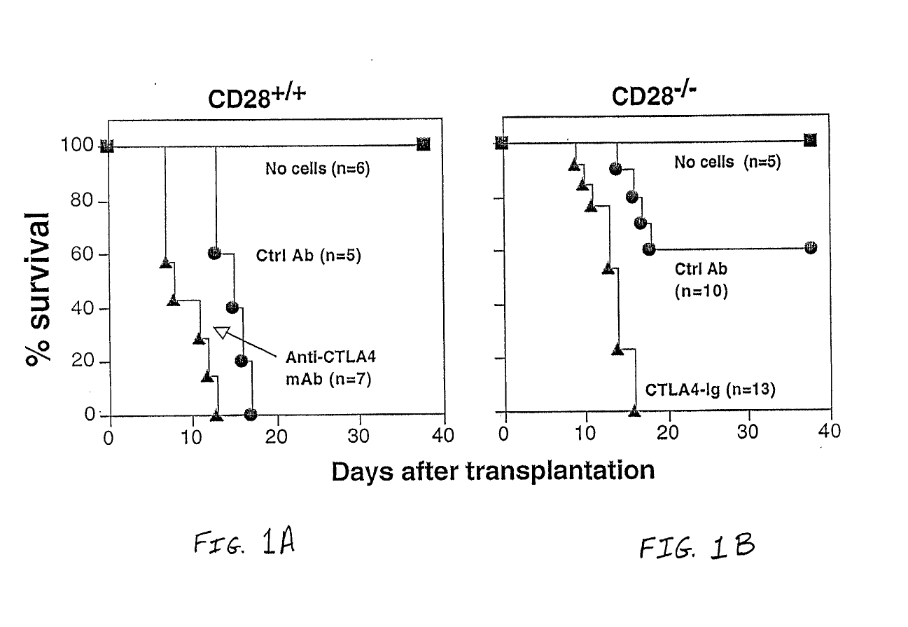

To determine the role of CTLA4-signals on the development of GVHD, the inventors first tested whether CTLA4 blockade with a nonstimulatory, bivalent mAb would accelerate GVHD. Sublethally irradiated (700 cGy) (B6×bm12)F1 mice were transplanted with purified CD4⁺ cells from wild type B6 mice and treated with anti-CTLA4 mAb or hamster IgG at 100 jig/dose every other day for a total of 8 doses. Treatment with anti-CTLA4 mAb was shown to accelerate GVHD lethality p=0.005) (FIG. 1A). To test whether CTLA4 function is dependent on CD28, sublethally irradiated (B6×bm12)F1 mice were transplanted with CD4⁺ cells from CD28^{-/-} B6 mice, and the recipients were treated with CTLA4-Ig or control L6-Ig. CTLA4-Ig-treatment significantly accelerated and exacerbated GVHD lethality compared with control treatment (p=0.00008) (FIG. 1B). These results indicate that the B7:CTLA4 interaction plays a protective role in the development of GVHD independent of CD28, and interfering with the B7:CTLA4 interaction enhances GVHD mortality.

### Example 3

### The effect of Anti-CD28 mAb in Preventing GVHD

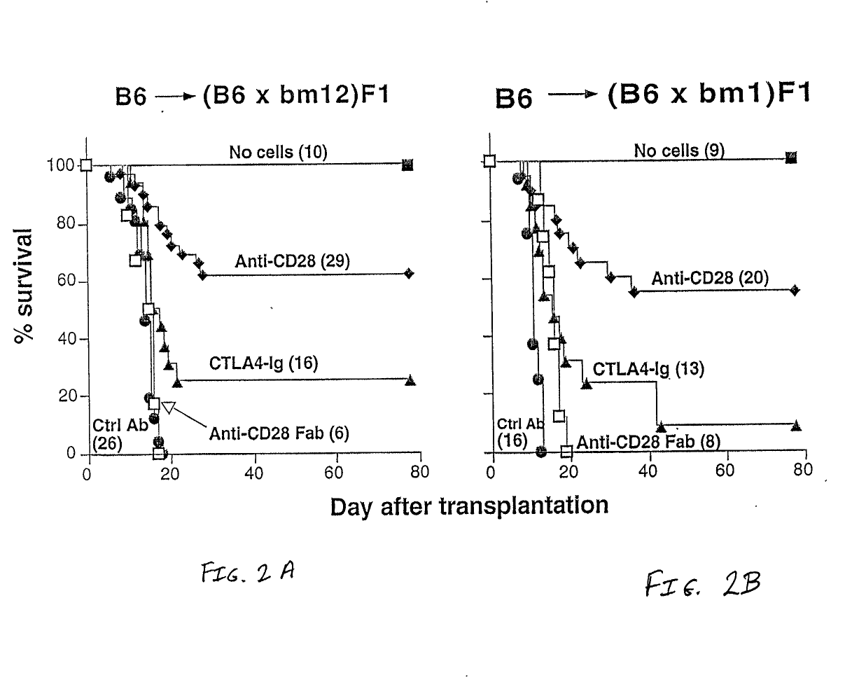

Blockage of B7 with soluble CTLA4-Ig or B7-specific mAbs can partially inhibit the development of GVHD in mice (Wallace et al, 1996; Blazar et al., 1994; Blazar et al., 1995; Blazar et al., 1996). Since CD28-signals enhance GVHD (Yu et al., 1998; Blazar et al., 1999), while CTLA4-signals inhibit GVHD, the inventors reasoned that the severity of GVHD would be decreased by selectively blocking CD28 costimulation while still allowing CTLA4 engagement on donor T cells. The inventors tested the effect of anti-CD28 mAb in preventing GVHD based on the observation that the administration of intact anti-CD28 mAb inhibits T cell expansion in vivo (Perez et al., 1997; Krunmmel et al., 1996; Zhang, 1996) despite anti-CD28 Abs induced T cell activation in vitro. Sublethally irradiated MHC class II incompatible (B6×bm12) or MHC class I incompatible (B6×bm1)F1 mice were transplanted with B6 CD4⁺ or CD8⁺ T cells respectively. Recipients were treated with anti-CD28 mAb, CTLA4-Ig or hamster IgG plus L6-Ig at 100 μg/dose from day 0 to day 14 every other day. Irradiation controls that were not transplanted developed transient pancytopenia, but all recovered and survived longer than 100 days. Recipients of allogenic T cells treated with control Abs became acutely ill with GVHD, characterized by progressive weight loss, ruffled fur and hunched back, and all died at a median of 15 days after transplant. Both CTLA4-Ig and anti-CD28 mAb improved survival compared with control Abs (p<0.0001), but anti-CD28 mAb was significantly more effective than CTLA4-Ig (p<0.01) (FIG. 2).

### Example 4

### The Effect of Anti-CD28 Fab

Monovalent CD28-specific Abs inhibit T cell activation and induce antigen-specific T cell energy in vitro (Martin et al., 1986; Tan et al., 1993). To test the hypothesis that treatment with anti-CD28 Fab fragments would prevent GVHD by blocking CD28-costimulation, (B6×bm12)F1 recipients of B6 CD4+T cells were injected with anti-CD28 Fab at 100 jg/dose once daily for a total of 16 doses. All the recipients treated with anti-CD28 Fab died within 18 days after transplant, while 75% of recipients treated with intact anti-CD28 mAb survived longer than 100 days (FIG. 2A), indicating that anti-CD28 mAb, at the dose and schedule administrated, did not have an effect on GVHD lethality.

Failure of anti-CD28 Fab in preventing GVHD lethality could be related to insufficient dose or infrequent administration, as Fab fragments have a shorter half life than intact Ab in vivo (Smith et al., 1976). The inventors increased the dose administrated to 200 μg/dose given once every 12 hours for a total of 32 doses. Flow cytometry analysis showed that the regimen was sufficient to maintain saturation of CD28 on peripheral blood T cells. The inventors chose to test such a regimen in B6→(B6×bm1)F1 transplants instead of B6→(B6×bm12)F1, because development of GVHD across MHC class I is more dependent on CD28 costimulation than GVHD across MHC class II (Yu et al., 1998). Death of recipients treated with anti-CD28 Fab was delayed 4-5 days, as compared to those treated with control Abs (FIG. 2B), but it was not prevented. These results indicated that anti-CD28 Fab administered at saturating doses for 16 days was not effective in preventing GVHD lethality.

### Example 5

### The Effects of Anti-CD28 mAb on Donor T Cell Activation and Expansion

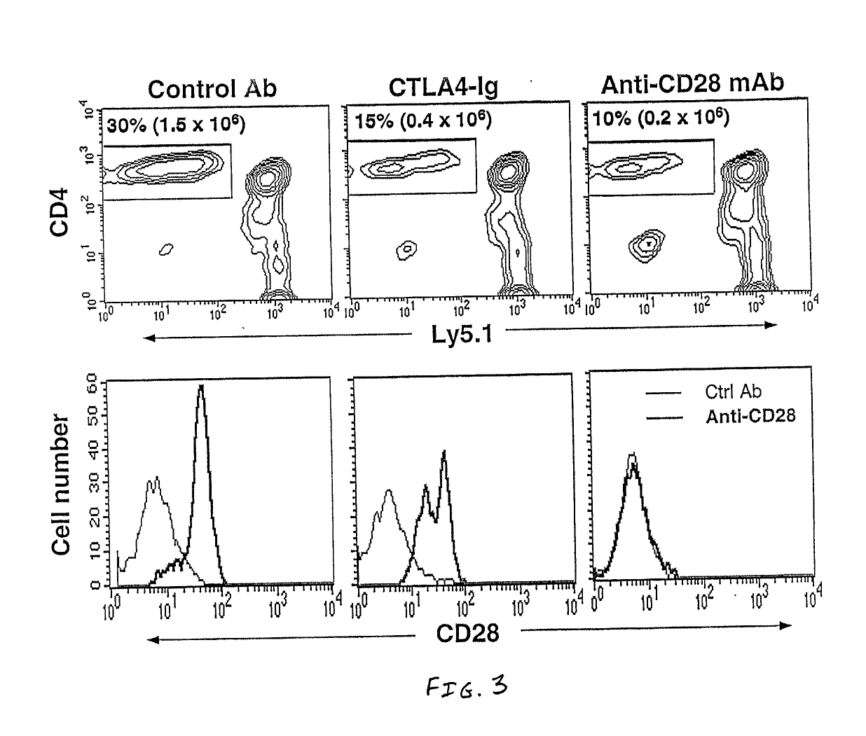

To elucidate the mechanisms by which anti-CD28-treatment prevents GVHD, we tested the effects of anti-CD28 mAb on donor T cell activation and expansion. Sublethally irradiated (B6.Ly5.1×bm12)F1 mice were transplanted with purified CD4 cells from B6.Ly5.2 donors and treated with anti-CD28 mAb, CTLA4-Ig or hamster Ig plus L6-Ig. Four days after transplantation, splenocytes from the recipient were stained for expression of CD4, Ly5.l, and CD28. The CD4⁺/Ly5.1⁻ phenotype identifies donor T cells. Donor CD4 T cells were much fewer in recipients treated with anti-CD28 mAb than in recipients treated with control Ab. CD28 expression on donor T cells was modulated by anti-CD28 mAb but not by control Abs or CTLA4-Ig (FIG. 3).

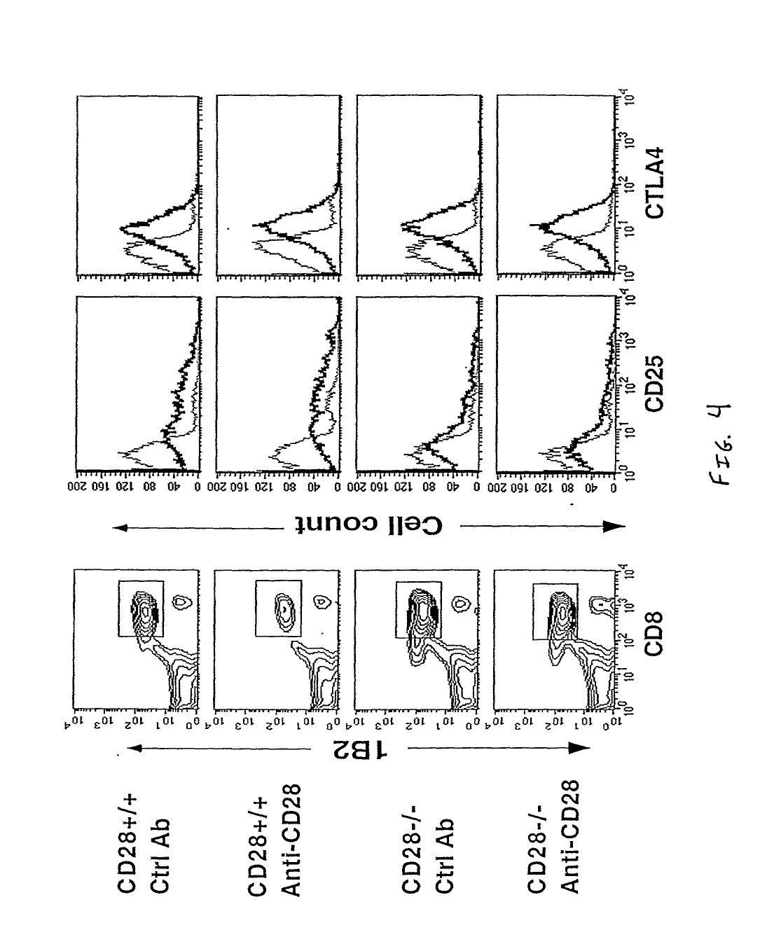

To follow the fate and function of alloreactive T cells and study the specificity of tolerance after transplantation, 2C TCR transgenic T cells were transplanted into CB6F1 recipients that expresses the specific alloantigen, Ld. In the CB6F1 recipients, 2C cells engraft, expand, become effectors and lead to extensive destruction of host B cells and double positive thymocytes (Yu et al., 1999). The inventors tested the effect of anti-CD28 mAb on activation of alloreactive 2C cells in CB6F1 recipients. Sublethally irradiated CB6F1 mice were transplanted with purified CD8⁺ cells from 2C wild type or 2C CD28−/− mice and treated with anti-CD28 mAb or hamster IgG. On day 4, 2C cells in recipient spleen were analyzed for expression CD25 and CTLA4 (FIG. 4). CTLA4 expression on 2C cells was independent of CD28 and was not affected by anti-CD28-treatment. CD25 expression was dependent of CD28, but it was not affected by anti-CD28-treatment. Results show that treatment with anti-CD28 mAb did not block early CD28 signaling that is required for CD25 expression, and did not interfere with CTLA4 expression.

### Example 6

### Effects of Anti-CD28 mAb on CD28⁺ T Cells in vivo

To determine whether anti-CD28 mAb prevents GVHD by depleting CD28⁺ T cells in vivo, the inventors transplanted purified CD8⁺ 2C T cells into irradiated CB6F1 or dm2B6F1 mice. The dm2B6F1 mice were used as non-allogenic recipients as controls, since dm2 is a BALB/c Ld loss mutant. Recipients were treated with anti-CD28 mAb or hamster IgG. On day 14, the inventors counted the numbers of 2C cells and B cells in the spleens of CB6F 1 and dm2B6F1 recipients. Treatment with anti-CD28 mnAb had no effect on 2C cells in dm2B6Fl recipients, indicating that this mAb did not deplete resting CD28⁺ cells in vivo.

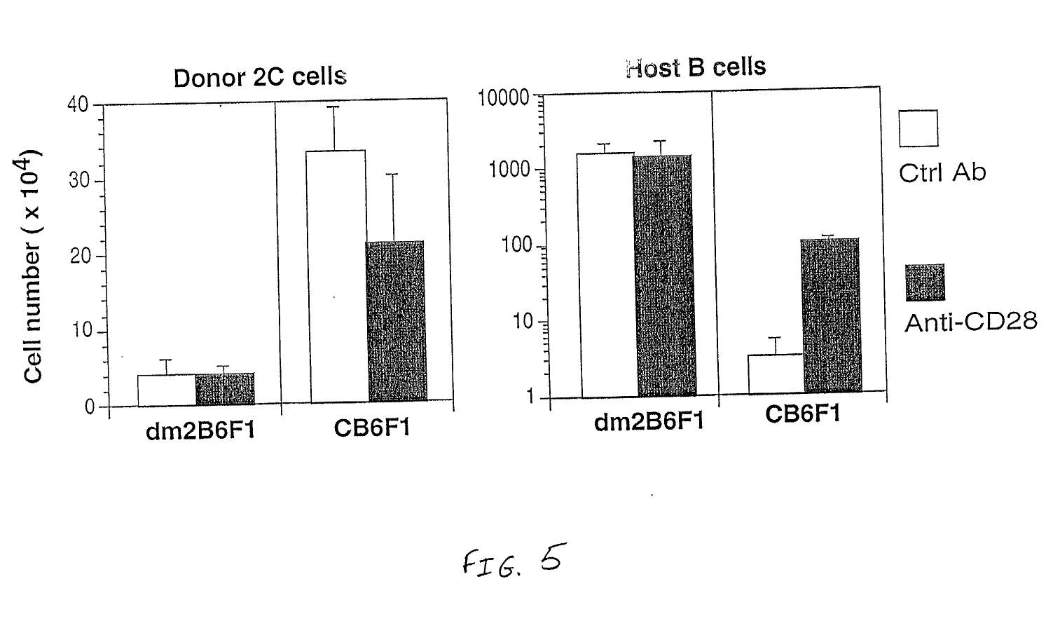

Treatment with anti-CD28 mAb decreased 2C population in CB6F1 recipient, indicating that the inhibitory effect of anti-CD28 mAb was specific for T cells that recognize alloantigens (FIG. 5A). The number of host B cells was 50-fold greater in CB6F 1 recipients treated with anti-CD28 mAb than in CB6F1 recipients treated with control Ab but 0.07-fold lower than in dm2B6F1 negative controls (FIG. 5B). These results indicate that treatment with anti-CD28 mAb did not abolish GVHD, but was able to decrease its intensity.

### Example 7

### Efficacy and Safety of Anti-CD28 mAb 9.3 as the Initial Treatment of Grades II-IV Acute GVHD After Transplantation of Allogeneic Hematopoietic Cells

In an open-label, prospective, non-randomized, single institution, phase II study patients with untreated grades II-IV acute GVHD receive mAb 9.3 daily for 14 days. The study consists of two treatment arms. Both include 9.3, but the approach depends on the prior GVHD prophylaxis regimen: Arm A enrolls 20 patients who have received a transplant from an HLA-compatible related or unrelated donor, with CSP alone or in combination with other agents; Arm B enrolls 20 patients who have received GVHD prophylaxis regimens not including CSP.

Patient Selection. Included are patients with untreated grades II-IV acute GVHD. Accrual of the first 10 patients is limited to HLA-compatible related or unrelated transplants.

Treatment Plan. After obtaining skin, gastrointestinal, or hepatic tissue samples for histopathological examination, patients with grades II-IV GVHD are entered on study. Patients receive an initial loading dose of mAb 9.3 0.4 mg/kg followed by 13 subsequent daily doses of 0.2 mg/kg. Patients may continue per assigned GVHD prevention protocol.

Study Endpoints. The safety of mAb 9.3 treatment is determined by the proportion of study patients who develop severe life-threatening acute reactions to mAb 9.3. Efficacy is determined by the proportion of patients who are alive 100 days after initiation of 9.3 therapy without further systemic treatment for acute GVHD. Patients with relapse of malignancy are censored from evaluation of GVHD at time of relapse.

Statistical Considerations. Treatment of acute GVHD with mnAb 9.3 is considered potentially efficacious and worthy of further studywith at least 80% confidence that the 100-day survival from the beginning of 9.3 therapy without further systemic treatment for acute GVHD is no less than 65%. This figure is based on the results of FHCRC protocol #573.2, a placebo-controlled study of Xomazyme plus prednisone for the primary treatment of acute GVHD. In that study, 65/114 (67%) patients treated with prednisone were alive 100 days from the beginning of treatment without the need for a second line of immunosuppressive therapy. Study Arms A and B are evaluated separately for the endpoints of efficacy. With 20 patients treated in each arm, one can be 80% confident that estimates of the success rate in each arm will be within at least 14% of the true success rate. Arm A of the study will be terminated for lack of adequate promise if 7 or more failures occur after 10 patients have been enrolled. The same considerations will be applied separately to arm B. This would allow at least 80% confidence that the true proportion of failures exceeds 50%.

### Example 8

### Prevention of GVHD with Anti-CD28 mAb 9.3 Administered Early After Transplantation

Patient Selection. The protocol enrolls patients eligible for related or unrelated marrow transplantation for treatment of any disease.

Treatment Regimen. Patients enrolled in this study receive a standard pretransplant conditioning regimen including CY (120 mg/kg) and 12-15 Gy fractionated TBI, or other agents, together with a standard posttransplant immunosuppressive regimen of CSP or rapamycin. Mab 9.3 is administered as a push on days 0-14 after transplantation.

Evaluation of Safety. Grading of severity are evaluated by the ward physicians. Grade I severity is defined as asymptomatic laboratory abnormalities or easily-tolerated symptoms that resolve after drug administration has been discontinued without requiring treatment. Grade II severity is defined as mild or tolerable symptoms having short duration and not interfering with normal activity and generally not requiring treatment. Grade III severity is defined as moderate or poorly tolerated, sustained symptoms which interfere with normal activity or require treatment but improve or resolve after drug administration has been discontinued or after treatment has been given. Grade IV severity is defined as intolerable, incapacitating, life-threatening or fatal symptoms not responding to treatment and resulting in permanent disability.

Evaluation of GVHD. The severity of acute GVHD is judged according to the standard Glucksberg criteria with allowances made for hepatic and gastrointestinal abnormalities caused by complications other than GVHD.

Rule for Escalation and Deescalation of Doses. Patients with acute leukemia have a 48% risk of grades III-IV GVHD after marrow transplantation from an HLA-A, B, DRBl-identical unrelated donor when MTX and CSP are given for prophylaxis. The goals of the phase I-II study are to identify a dose of mAb 9.3 where the risk of grades III-IV GVHD is 15-25% or less and the incidence of grade III toxicity related to mAb 9.3 administration is 20% or less with no grade IV toxicity. Based on prior experience, it is expected that the maximum-tolerated total dose of mAb 9.3 will be close to 1 g/m² when administered as a push. Therefore, mAb 9.3 at four total dose levels: 0.001, 0.01, 0.1 and 1.0 g/m² is tested. Enrollment begins at dose-level 1, and dose levels are escalated or deescalated according to rules described in Table 3. The relatively low dose of 0.001 mg/m² is based on observations that this dose was well tolerated in patients with Hodgkin's Disease. It remains to be seen whether the immunotoxin will cause more toxicity when administered shortly after the marrow transplant conditioning regimen.

TABLE 3
Rules for Escalation or Deescalation of mAb 9.3 Dose

| Event | Limit | Action |
|---|---|---|
| Phase I | | |
| Grades III-IV GVHD | 1 at dose x | Continue phase I at |
| | | dose x × 1; |
| | | if dose x = 4, |
| | | then begin phase II. |
| Toxicity | 1 grade IV or | Begin phase II at |
| | 4/4-8 grade III | dose x − 1; |
| | | if dose x = 0, |
| | | then terminate trial. |
| No limit reached with | | Begin phase II at dose x. |
| n = 8 at dose x | | |
| Phase II | | |
| Grades III-IV GVHD | 4/4-9, 5/11-12, | If dose level x = 4 |
| | 6/14-16, 7/18-20 | or if limits for |
| | at dose x | toxicity have already |
| | | been reached at dose |
| | | x + 1, then terminal |
| | | trial. Otherwise, restart |
| | | phase II at dose x + 1. |
| Toxicity | 1 grade IV or 4/4-10, | If dose level x = 0 |
| | 5/12-14, or | or if limits for GVHD |
| | 6/16-20 grade III | have already been |
| | at dose x | reached at dose |
| | | x − 1, then terminal |
| | | trial. Otherwise, restart |
| | | phase II at dose x − 1. |
| No limit reached with | | Terminate trial. |
| n = 20 at dose x | | |

In phase II, “n” includes all patients enrolled at dose x from both phase I and phase II.

The primary goal of the phase I part of the study is to determine the maximum dose (up to 9 mg/m²) that can be tolerated without exceeding the toxicity limits described above. These toxicity limits were developed by using the lower limit of the 80% confidence interval as an approximate guide. If these limits for toxicity are not exceeded and if grade III or IV GVHD does not occur in any of 8 patients enrolled at a given dose, there would be 90% confidence that the true risk of GVHD does not exceed 25%, and the phase II part of the study would be initiated. The primary goal of this phase is to determine if a dose associated with 15-25% or lower risk of grades III-IV GVHD and <20% risk of toxicity can be identified, using the lower limit of the 80% confidence intervals as an approximate guide for stopping the study because of an excessive incidence of GVHD or toxicity.

Pharmacokinetic Studies. A double determinant-sandwich radioimmunoassay can detect mAb 9.3 at concentrations as low as 1 ng/mL. Peak and trough serum concentrations can be measured, and VD, Trip and AUC estimated from the data. Clinical correlations are evaluated primarily for associations with the occurrence and severity of GVHD.

### Example 9

### The effect of Anti-human CD28 in Preventing GVHD Caused by Human T Cells in Immunodeficient Mice

The inventors tested the effect of anti-human CD28 antibody 9.3 in preventing GVHD caused by human T cells in non-obese diabetic (NOD)/severe combined immune deficient (SCID) mice. 9.3 is a murine IgG2a monoclonal antibody (mnAb) specific for human CD28 (ref.1). NOD/SCID mice received whole body irradiation with 250 cGy. Peripheral blood mononuclear cells (PBMC) from normal human volunteers were transplanted at the dose of 300 million per mouse into the peritoneal cavity of each irradiated NOD/SCID mouse. Control mice received irradiation but no human cells. One group of mice was transplanted with human PBMC and treated with anti-CD28 mAb 9.3 at the dose of 100 μg IP from day 0 to day 14. One group of mice was transplanted with human PBMC and control vehicle. All 11 irradiation controls survived. Seven of 8 mice transplanted with human PBMC and treated with vehicle died (p=0.001), as consequence of GVHD. In contrast, all 11 mice who received human PBMC and were treated with anti-CD28 mAb 9.3 survived (p=0.001). These results demonstrated that anti-human CD28 mAb 9.3 is effective in the prevention of GVHD mediated by human T cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

U.S. Pat. No. 4,016,100

U.S. Pat. No. 4,089,801

U.S. Pat. No. 4,196,265

U.S. Pat. No. 4,234,871

U.S. Pat. No. 4,485,054

U.S. Pat. No. 4,554,101

U.S. Pat. No. 5,821,333

U.S. Pat. No. 5,869,451

U.S. Pat. No. 5,888,773

U.S. Pat. No. 5,889,157

U.S. Pat. No. 5,933,819

U.S. Pat. No. 5,525,503

Antibodies: A Laboratory Manual, eds., Harlow and Lane, Cold Spring Harbor Laboratory, N.Y., 1988;

Armitage, “Bone marrow transplantation,” New England J. of Med., 330:827, 1994.

Ballas, Rasmussen, Krieg, “Induction of NK activity in murine and human cells by CpG motifs in oligodeoxynucleotides and bacterial DNA,” J. Immunology, 157:1840-1845, 1996.

Bammel, Brand, Germon, Smith, “Interaction of the extrinsic potential-sensitive molecular probe diS-C3-(5) with pigeon heart mitochondria under equilibrium and time-resolved conditions,” Arch. Biochem. Biophys., 244:67-84, 1986.

Bangham et al., J. Mol. Biol. 13:238-252, 1965.

Blazar, Sharpe, Taylor, Panoskaltsis-Mortari, Gray, Korngold, Vallera, “Infusion of anti-B7.1 (CD80) and anti-B7.2 (CD86) monoclonal antibodies inhibits murine graft-versus-host disease lethality in part via direct effects on CD4+ and CD8+ T cells,” J. of Immun., 157:3250, 1996.

Blazar, Taylor, Linsley, Vallera, “In vivo blockade of CD28/CTLA4: B7/BB1 interaction with CTLA4-Ig reduces lethal murine graft-versus-host disease across the major histocompatibility complex barrier in mice,” Blood, 83:3815, 1994.

Blazar, Taylor, Panoskaltsis-Mortari, Gray, Vallera, “Coblockade of the LFA1:ICAM and CD28/CTLA4:B7 pathways is a highly effective means of preventing acute lethal graft-versus-host disease induced by fully major histocompatibility complex-disparate donor grafts,” Blood, 85:2607, 1995.

Blazar, Taylor, Panoskaltsis-Mortari, Sharpe, Vallera, “Opposing roles of CD28:B7 and CTLA-4:B7 pathways in regulating in vivo alloresponses in murinerecipients of -MHC disparate T cells,” J. of Immun., 162:6368, 1999.

Brown, “Fully automated baseline correction of ID and 2D NMR spectra using bernstein polynomials,” Magn. Reson., Series A 114:268-270, 1995.

Buhlmann, Gonzalez, Ginther, Panoskaltsis, Blazar, Greiner, Rossini, Flavell, Noelle, “Cutting edge: sustained expansion of CD8+T cells requires CD 154 expression by T cells in acute graft versus host disease,” J. of Immun., 162:4373, 1999.

Calvo, Amsen, Kruisbeek, “Cytotoxic T lymphocyte antigen 4 (CTLA-4) interferes with extracellular signal-regulated kinase (ERK) and Jun NH2-terminal kinase (JNK) activation, but does not affect phosphorylation of T cell receptor zeta and ZAP70,” J. of Exper. Med., 186:1645, 1997.

Chaplin et al., Immunogenetics, 49:583-584, 1999.

Chou and Fasman, “Conformational Parameters for Amino Acids in Helical, β-Sheet, and Random Coil Regions Calculated from Proteins,” Biochemistry, 13(2):211-222, 1974b.

Chou and Fasman, “Empirical Predictions of Protein Conformation,” Ann. Rev. Biochem., 47:251-276, 1978b.

Chou and Fasman, “Prediction of b-Turns,” Biophys. J, 26:367-384, 1979.

Chou and Fasman, “Prediction of Protein Conformation,” Biochemistry, 13(2):222-245, 1974a.

Chou and Fasman, “Prediction of the Secondary Structure of Proteins from Their Amino Acid Sequence,” Adv. Enzymol. Relat. Areas Mol. Biol., 47:45-148, 1978a.

Clark and Dalman, Immunogenetics, 35:54-57, 1992.

de Duve, C.; de Barsy, T.; Poole, B.; Trouet, A.; Tulkens, P.; van Hoof, F. Biochem. Pharm. 1974, 23.

Deamer and Uster, “Liposome Preparation: Methods and Mechanisms,” In: Liposomes, M. Ostro (Ed.), 1983

Drug Carriers In Biology and Medicine, G. Gregoriadis ed. (1979) pp. 287-341.

Duve, Barsy, Poole, Trouet, Tulkens, van Hoof, “Lysosomotropic agents,” Biochem. Pharm, 23, 1974.

Ellena, Dominey, Archer, Xu, Cafiso, “Localization of hydrophobic ions in phospholipid bilayers using 1H nuclear Overhauser effect spectroscopy,” Biochem., 26:4584-4592, 1987.

Fairlie, West, Wong, “Towards protein surface mimetics,” Curr Med Chem., 5(1):29-62, 1998.

Fallarino, Fields, Gajewski, “B7-1 engagement of cytotoxic T lymphocyte antigen 4 inhibits T cell activation in the absence of CD28,” J. of Exper. Med., 188:205, 1998.

Fitch, Yunis, Chevli, Gonzalez, “High affinity accumulation of chloroquine by mouse erythrocytes infected with Plasmodium berghei,” J. Clin. Invest., 54:24-33, 1974.

Fox, “Mechanism of action of hydroxychloroquine as an antirheumatic drug,” Sem. Arthritis Rheumatism, 23:82-91, 1993.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gray, “Linear prediction guide,” Magn. Moments, 7:30-33, 1990.

Gross et al., J. Immunology, 144:3201-3210, 1990.

Hansen, Gooley, Martin, Appelbaum, Chauncey, Clift, Petersdorf, Radich, Sanders, Storb, Sullivan, Anasetti, “Bone marrow transplants from unrelated donors for patients with chronic myeloid leukemia,” New England J. of Med., 338:962, 1998.

Hansen, Petersdorf, Martin, Anasetti, “Hematopoietic stem cell transplants from unrelated donors,” Immunological Reviews, 157:141, 1997.

Hara, Fu, Hansen, “Human T cell activation. II. A new activation pathway used by a major T cell population via a disulfide-bonded dimer of a 44 kilodalton polypeptide (9.3 antigen),” J. of Exper. Med., 161:1513, 1985.

Holdorf, et al. Proline residues in CD28 and the Src homology (SH)3 domain of Lck are required for T cell costimulation. J. Exp Med, 190(3):375-84 1999.

Ismail, Dascombe, Carr, North, “An exploration of the structure-activity relationships of 4-aminoquinolines: novel antimalarials with activity in-vivo,” J. Pharm. Pharmnacol., 48:841-850, 1996.

Isono and Seto, Immunogenetics, 42:217-220, 1995.

Krieg et al., “Lymphocyte activation by CpG dinucleotide motifs in prokaryotic DNA,” Trends Microbiol., 4(2):73-6, 1996.

Krieg, Matson, Fisher, “Oligodeoxynucleotide modifications determine the magnitude of B cell stimulation by CpG motifs,” Antisense Nucleic Acid Drug Dev., 6(2):133-9, 1996.

Krieg, Yi, Matson, Waldschmidt, Bishop, Teasdale, Koretzky, Klinman, “CpG motifs in bacterial DNA trigger direct B-cell activation,” Nature, 374:546-549, 1995.

Krummel and Allison, “CD28 and CTLA-4 have opposing effects on the response of T cells to stimulation [see comments],” J. of Exper. Med., 182:459, 1995.

Knumnel, Sullivan, Allison, “Superantigen responses and co-stimulation: CD28 and CTLA-4 have opposing effects on T cell expansion in vitro and in vivo,” Intl. Immun., 8:519, 1996.

Larsen, Elwood, Alexander, Ritchie, Hendrix, Tucker-Burden, Cho, Aruffo, Hollenbaugh, Linsley, Winn, Pearson, “Long-term acceptance of skin and cardiac allografts after blocking CD40 and CD28 pathways,” Nature, 381:434, 1996.

Leach, Krummel, Allison, “Enhancement of antitumor immunity by CTLA-4 blockade,” Science, 271:1734, 1996.

Lee, Chuang, Griffn, Khattri, Hong, Zhang, Straus, Samelson, Thompson, Bluestone, “Molecular basis of T cell inactivation by CTLA-4,” Science, 282:2263, 1998.

Lee et al., J. Immunology 145:344-352, 1990.

Lenschow, Walunas, Bluestone, “CD28/B7 system of T cell costimulation,” Ann. Rev. of Immun., 14:233, 1996.

Lin, Rathmell, Gray, Thompson, Leiden, Alegre, “Cytotoxic T lymphocyte antigen 4 (CTLA4) blockade accelerates the acute rejection of cardiac allografts in CD28-deficient mice: CTLA4 can function independently of CD28,” J. of Exper. Med., 188:199, 1998.

Linsley, et al., CD28/CTLA-4 receptor structure, binding stoichiometry and aggregation during T-cell activation, Res Immunol. 146(3): 130-40, 1995.

Linsley, Brady, Urnes, Grosmaire, Damle, Ledbetter, “CTLA-4 is a second receptor for the B cell activation antigen B7,” J. of Exper. Med., 174:561, 1991.

Macfarlane and Manzel, “Antagonism of immunostimulatory CpG—oligodeoxynucleotides by quinacrine, chloroquine and structurally related compounds,” J. Immunol., 160:1122-1131, 1998.

Macfarlane, Manzel, Krieg, “Unmethylated CpG-containing oligodeoxynucleotides inhibit apoptosis in WEHI 231 B-lyrnphocytes induced by several agents: evidence for blockade at a distal signaling step,” Immunology, 91:586-593, 1997.

Martin P J. Hansen J A. Nowinski R C. Brown M A. A new human T-cell differentiation antigen: unexpected expression on chronic lymphocytic leukemia cells. Immunogenetics. 11(5):429-39, 1980.

Martin, Ledbetter, Morishita, June, Beatty, Hansen, “A 44 kilodalton cell surface homodimer regulates interleukin 2 production by activated human T lymphocytes,” J. of Immun., 136:3282, 1986.

Miller, Vanderlugt, Lenschow, Pope, Karandikar, Dal Canto, Bluestone, “Blockade of CD28/B7-1 interaction prevents epitope spreading and clinical relapses of murine EAR,” Immunity, 3:739,1995.

Mokrosz, Duszynska, Strekowski, Pharmazie, 538, 1992.

Moore, “Designing peptide mimetics,” Trends Pharmacol Sci., 15(4):124-9, 1994.

Mullis, Faloona, Scharf, Saiki, Horn and Erlich, “Specific enzymatic amplification of DNA in vitro: the polymerase chain reaction,” Biotechnology, 24:17-27, 1992.

Mullis, “Target amplification for DNA analysis by the polymerase chain reaction,” Ann. Biol. Clin. (Paris); 48:579-82, 1990.

Ohkuma and Poole, “Cytoplasmic vacuolation of mouse peritoneal macrophages and the uptake into lysosomes of weakly basic substances,” J. Cell. Biol., 90:656-664, 1981.

Ohkuma and Poole, “Fluorescence probe measurements of the intralysosomal pH in living cells and the perturbation of pH by various agents,” Proc. Natl. Acad. Sci. USA, 75:3327-3331, 1978.

Parsons et al., Immungenetics, 43:388-391, 1996.

Pastori et al., Immunogenetics, 39, 1994.

Perez, Van Parijs, Biuckians, Zheng, Strom, Abbas, “Induction of peripheral T cell tolerance in vivo requires CTLA4 engagement,” Immunity, 6:411, 1997.

Perrin, Maldonado, Davis, June, Racke, “CTLA-4 blockade enhances clinical disease and cytokine production during experimental allergic encephalomyelitis,” J. of Immun., 157:1333, 1996.

Remington's Pharmaceutical Sciences, 15th ed., Mack Publishing Company, Easton, Pa., 1980.

Saigo and Ryo, “Therapeutic Strategy for post-transfusion graft-versus-host disease,” Int. J. Hematol., 69:147-151, 1999.

Saito, Sakurai, Ohata, Kohsaka, Hashimoto, Okumura, Abe, Azuma, “Involvement of CD40 ligand-CD40 and CTLA4-B7 pathways in murine acute graft-versus-host disease induced by allogeneic T cells lacking CD28,” J. of Immun., 160:4225, 1998.

Shahinian, Pfeffer, Lee, Kundig, Kishihara, Wakeham, Kawai, Ohashi, Thompson, Mak, “Differential T cell costimulatory requirements in CD28-deficient mice,” Science, 261:609, 1993.

Smith, Haber, Yeatman, Butler, Jr., “Reversal of advanced digoxin intoxication with Fab fragments of digoxin-specific antibodies,” New England J. of Med., 294:797, 1976.

States, Haberkom, Rubin, “A two-dimensional nuclear overhauser experiment with pure absorption phase in four quadrants,” Magn. Reson., 48:286-292, 1982.

Strekowski, Kiselyov, Hojjat, “The o-amino-trifluoromethyl functionality as a novel synthon for 4-fluoroquinolines,” J. Org. Chem., 591(994)5886-5890, 1994.

Strekowski, L.; Gulevich, Y.; Baranowski, T. C.; Parker, A. N.; Kiselyov, A. S.; Lin, S.- Y.; Tanious, F. A.; Wilson, W. D. J. Med. Chem., 39, 3980, 1996.

Strekowski, L.; Janda, L.; Patterson, S. E.; Nguyen, J. Tetrahedron, 52, 3273, 1996.

Strekowski, L.; Zegrocka, O.; Windham, C.; Czarny, A. Org. Process Res. Dev., 1, 384, 1997.

Strekowski, Patterson, Janda, Wydra, Harden, Lipowska, Cegl, “Further studies on the cyclization of aromatic azaomethines ortho-substituted with a trifluoromethyl group: Synthesis of 2,4-di- or 2,3,4-trisubstituted quinolines,” Org. Chem., 57, 1992.

Strekowski, Wilson, Mokrosz, Mokrosz, Harden, Tanious, Wydra, Crow, Jr., “Quantitative structure-activity relationship analysis of cation-substituted polyaromatic compounds as potentiators (amplifiers) of bleomycin-mediated degradation of DNA,” J. Med. Chem., 34:580-588, 1991.

Szoka and Papahadjopoulos, Proc. Natl. Acad. Sci. USA, 75: 4194-4198, 1978.

Tan, Anasetti, Hansen, Melrose, Brunvand, Bradshaw, Ledbetter, Linsley, “Induction of alloantigen-specific hyporesponsiveness in human T lymphocytes by blocking interaction of CD28 with its natural ligand B7/BB1,” J. of Exper. Med., 177:165, 1993.

Thompson and Allison, “The emerging role of CTLA-4 as an immune attenuator,” Immunity, 7:445, 1997.

Tivol, Borriello, Schweitzer, Lynch, Bluestone, Sharpe, “Loss of CTLA4 leads to massive lymphoproliferation and fatal multiorgan tissue destruction, revealing a critical negative regulatory role of CTLA-4,” Immunity, 3:541, 1995.

Tonkinson and Stein, “Patterns of intracellular compartmentalization, trafficking and acidification of 5′-fluorescein labeled phosphodiester and phosphorothioate oligodeoxynucleotides in HL60 cells,” Nucleic Acid Res., 22:4268-4275, 1994.

Wallace, “Antimalarial agents and lupus,” Rheumatic Disease Clinics of Portly America, 20:243-263, 1994.

Wallace, Johnson, MacMaster, Kennedy, Gladstone, Linsley, “CTLA4Ig treatment ameliorates the lethality of murine graft-versus-host disease across major histocompatibility complex barriers,” Transplantation, 58:602, 1996.

Walunas, Lenschow, Bakker, Linsley, Freeman, Green, Thompson, Bluestone, “CTLA-4 can fuinction as a negative regulator of T cell activation,” Immunity. 1:405, 1994.

Waterhouse, Penninger, Timms, Wakeham, Shahinian, Lee, Thompson, Griesser, Mak, “Lymphoproliferative disorders with early lethality in mice deficient in Ctla-4,” Science, 270:985, 1995.

Wawrzynczak & Thorpe “Methods for Preparing Immunotoxins: Effect of the Linkage on Activity and Stability”, In: Immunoconjugates: Antibody Conjugates in Radioimaging and Therapy of Cancer, Vogel (ed.), New York, Oxford University Press, pp. 28-55, 1987.

Wilson, 1998. “DNA and RNA intercalators,” In: DNA and aspects of molecular biology, Kool (ed.), Vol. 7, 1998.

Wilson, W. D.; Ratmeyer, L.; Zhao, M.; Strekowski, L.; Boykin, D. Biochemistry 1993, 32, 4098.

Yi, Hornbeck, Lafrenz, Krieg, “CpG DNA rescue of murine B lymphoma cells from anti-IgM induced growth arrest and programmed cell death is associated with expression of c-myc, c-myb, myn, and bc1-2,” J. Immunol., 157:4918-4925, 1996.

Yi, Tuetken, Redford, Waldschlnidt, Kirsch, Krieg, “CpG motifs in bacterial DNA activate leukocytes through the pH-dependent generation of reactive oxygen species,” J. Immunol., 160:4755-4761, 1998.

Yu, Martin, Anasetti, “Role of CD28 in acute graft-versus-host disease,” Blood, 92:2963, 1998.

Zhang, “The fate of adaptively transferred antigen-specific T cells in vivo,” European J. of Immun., 26:2208, 1996.

What is claimed is:

1. A method of inhibiting an immune response comprising administering to a subject an effective amount of a purified anti-CD28 antibody preparation, wherein said preparation modulates the CD28 receptor thereby inhibiting said immune response.

2. The method of claim 1, wherein inhibiting said immune response is by reversing T cell activation.

3. The method of claim 1, wherein inhibiting said immune response is by blocking T cell activation.

4. The method of claim 1, wherein said antibody preparation is polyclonal.

5. The method of claim 4, wherein said antibody preparation is monoclonal.

6. The method of claim 5, wherein said antibody is monovalent.

7. The method of claim 5, wherein said antibody is bivalent.

8. The method of claim 5, wherein said antibody is human.

9. The method of claim 5, wherein said antibody is chimeric.

10. The method of claim 9, wherein said chimeric antibody is humanized.

11. The method of claim 10, wherein said humanized antibody comprises mammalian variable chain regions and human constant chain regions.

12. The method of claim 11, wherein said mammalian variable chain regions are selected from the group consisting of mouse, rat, hamster, monkey, goat and human

13. The method of claim 1, wherein said subject is susceptible to graft-versus-host disease, marrow transplant rejection, organ transplant rejection or tissue transplant rejection.

14. The method of claim 13, wherein said subject has graft-versus-host disease.

15. The method of claim 1, wherein said subject has an autoimmune disease.

16. The method of claim 15, wherein said autoimmune disease is psoriasis, diabetes mellitus, multiple sclerosis, rheumatoid arthritis, systemic sclerosis, systemic lupus erythematosus, dermatomyositis, polymyositis, Sjogren syndrome, polyarteritis nodosa, or vasculitis.

17. The method of claim 1, wherein said administering is by injection.

18. The method of claim 17, wherein said injection is performed local or regioril to the site of said immune response.

19. The method of claim 18, wherein said injection site is further defined as thymus, spleen, lymph nodes, bone marrow, tonsils, adenoids or blood stream.

20. The method of claim 17, wherein said injection is parenteral, intravenous, intramuscular, subcutaneous, intradermal or intraperitoneal.

21. The method of claim 20, wherein said injection is intraperitoneal or intravenous.

22. The method of claim 17, further comprising multiple injections.

23. The method of claim 22, wherein injections are performed at the same time at different locations.

24. The method of claim 22, wherein injections are performed at different times.

25. The method of claim 20, wherein said injection is via continuous infusion.

26. The method of claim 1, wherein said method further comprises administering an immunosuppressive agent.

27. The method of claim 26, wherein said immunosuppressive agent is selected from the group consisting of azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody.

28. A method of inhibiting an immune response in a subject comprising the steps of:
(i) obtaining lymphocyte cells from said subject;
(ii) contacting said lymphocyte cells with an anti-CD28 antibody preparation; and
(iii) administering said contacted cells to said subject,
wherein said preparation reverses T cell activation thereby inhibiting said immune response.

29. The method of claim 28, wherein said antibody preparation is polyclonal.

30. The method of claim 29, wherein said antibody preparation is monoclonal.

31. The method of claim 30, wherein said antibody is monovalent.

32. The method of claim 30, wherein said antibody is bivalent.

33. The method of claim 30, wherein said antibody is human.

34. The method of claim 30, wherein said antibody is chimeric.

35. The method of claim 34, wherein said chimeric antibody is humanized.

36. The method of claim 35, wherein said humanized antibody comprises mammalian variable chain regions and human constant chain regions.

37. The method of claim 36, wherein said mammalian variable chain regions are selected from the group consisting of mouse, rat, hamster, monkey, goat and human.

38. The method of claim 28, wherein said subject is susceptible to graft-versus-host disease, marrow transplant rejection, organ transplant rejection or tissue transplant rejection.

39. The method of claim 38, wherein said subject has graft-versus-host disease.

40. The method of claim 28, wherein said subject has an autoimmune disease.

41. The method of claim 40, wherein said autoimmune disease is psoriasis, diabetes mellitus, multiple sclerosis, rheumatoid arthritis, systemic sclerosis, systemic lupus erythematosus, dermatomyositis, polymyositis, Sjogren syndrome, polyarteritis nodosa, or vasculitis.

42. The method of claim 28, wherein said administering is by injection.

43. The method of claim 28, wherein said lymphocyte cells are obtained from thymus, spleen, lymph nodes, bone marrow, tonsils, adenoids or blood stream.

44. The method of claim 42, wherein said injection further comprises an immunosuppressive agent.

45. The method of claim 44, wherein said immunosuppressive agent is selected from the group consisting of azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody.

46. A method of inhibiting an immune response comprising administering to a subject an effective amount of a CD28 ligand, wherein said preparation modulates the CD28 receptor thereby inhibiting said immune response.

47. The method of claim 46, wherein inhibiting said immune response is by reversing T cell activation.

48. The method of claim 46, wherein inhibiting said immune response is by blocking T cell activation.

49. The method of claim 46, wherein said ligand is an antibody.

50. The method of claim 46, wherein said subject is susceptible to graft-versus-host disease, marrow transplant rejection, organ transplant rejection or tissue transplant rejection.

51. The method of claim 50, wherein said subject has graft-versus-host disease.

52. The method of claim 46, wherein said subject has an autoimmune disease.

53. The method of claim 52, wherein said autoimmune disease is psoriasis, diabetes mellitus, multiple sclerosis, rheumatoid arthritis, systemic sclerosis, systemic lupus erythematosus, dermatomyositis, polymyositis, Sjogren syndrome, polyarteritis nodosa, or vasculitis.

54. The method of claim 46, wherein said administering is by injection.

55. The method of claim 54, wherein said injection is performed local or regional to the site of said immune response.

56. The method of claim 55, wherein said injection site is further defined as thymus, spleen, lymph nodes, bone marrow, tonsils, adenoids or blood stream.

57. The method of claim 54, wherein said injection is parenteral, intravenous, intramuscular, subcutaneous, intradermal or intraperitoneal.

58. The method of claim 57, wherein said injection is intraperitoneal or intravenous.

59. The method of claim 54, further comprising multiple injections.

60. The method of claim 59, wherein injections are performed at the same time at different locations.

61. The method of claim 59, wherein injections are performed at different times.

62. The method of claim 57, wherein said injection is via continuous infusion.

63. The method of claim 57, wherein said injection further comprises an immunosuppressive agent.

64. The method of claim 63, wherein said immunosuppressive agent is selected from the group consisting of azathioprine, tacrolimus, sirolimus, rapamycin, thalidomide, leflunomide, clofazimine, mycophenolic acid, fludarabine, guanosine arabinoseide, cytosine arabinoseide, cyclosporins, prednisone, antithymocyte globulins, cyclophosphamide, glucocorticoids, methotrexate, anti-CD40 ligand antibody, anti-CD40 antibody, anti-CD3 antibody, anti-CD25 antibody, anti-CD30 antibody and anti-OX40 antibody.

65. A method of inhibiting an immune response comprising administering to a subject an effective amount of a ligand, wherein said ligand blocks CD28 signal transduction thereby inhibiting said immune response.

66. The method of claim 65, wherein said ligand binds PI 3-kinase.
